SEQUENCE LISTING (11 sequences)

SEQ ID NO: 1 - protein, 14 aa, Artificial Sequence
  HBP NCAM
KGRDVILKKDVRFI

SEQ ID NO: 2 - protein, 14 aa, Artificial Sequence
  HBP mutant (peptide M)
KGRDVILAKDVRVI

SEQ ID NO: 3 - protein, 14 aa, Artificial Sequence
  HBP mutant (peptide M1n)
KGRDVILNNDVRFI

SEQ ID NO: 4 - protein, 14 aa, Artificial Sequence
  HBP mutant (peptide M3n)
KGRDVILNNQVRFI

SEQ ID NO: 5 - protein, 14 aa, Artificial Sequence
  HBP mutant (peptide M4)
AGRDVILNNDVRFI

SEQ ID NO: 6 - protein, 14 aa, Artificial Sequence
  HBP mutant (peptide M5)
NGRDVILKKDVLFI

SEQ ID NO: 7 - protein, 14 aa, Artificial Sequence
  HBP mutant (peptide M6)
NGLDVILIIDVRFI

SEQ ID NO: 8 - protein, 14 aa, Artificial Sequence
  NGF fragment
KGKEVMVLGEVNIN

SEQ ID NO: 9 - protein, 14 aa, Artificial Sequence
  NT-3 fragment
RGHQVTVLGEIKTG

SEQ ID NO: 10 - protein, 14 aa, Artificial Sequence
  NT-4/5 fragment
RGREVEVLGEVPAA

SEQ ID NO: 11 - protein, 14 aa, Artificial Sequence
  BDNF fragment
SGGTVTVLEKVPVS

(19) United States
(12) Patent Application Publication
(10) Pub. No.: US 2008/0226639 A1
(43) Pub. Date: Sep. 18, 2008

(54) Heparin Binding Peptide

(75) Inventors: Elisabeth Bock, Charlottenlund (DK); Vladimir Berezin, Copenhagen N (DK)

Correspondence Address: BROWDY AND NEIMARK, P.L.L.C.;624 NINTH STREET, NW, SUITE 300, WASHINGTON, DC, 20001-5303, US

(73) Assignee: Enkan Pharmaceuticals A/S, Copenhagen (DK)

(21) Appl. No.: 11/629,077

(22) PCT Filed: Jun. 10, 2005
(86) PCT No.: PCT/DK2005/000382

§ 371 Date: Dec. 19, 2007

(30) Foreign Application Priority Data
Oct. 6, 2004 (DK) PA 2004 00909

(51) Int. Cl. A61K 39/395; C07K 7/00; C07K 2/00; A61K 38/03; C07K 16/44; A61P 35/00; A61P 3/10; A61P 25/28

(52) U.S. Cl. 424/139.1; 530/329; 530/328; 530/327; 530/324; 530/387.1; 514/17; 514/16; 514/15; 514/14

(57) ABSTRACT

The invention relates to new peptide fragments capable of stimulating neurite outgrowth, stimulating of cell survival, stimulating neural plasticity associated with memory and learning, and modulating cell motility. The biological activity of the peptide fragments is associated with their capability of binding and activating a neurotrophin receptor of the Trk family. The peptide fragments of the invention comprise an amino acid motif which is essential for binding and activating a neurotrophin receptor of the Trk family. The invention also concerns pharmaceutical compositions comprising the compounds and uses thereof for prevention and/or treatment of conditions and/or diseases, wherein neurotrophing, Tkr receptors and/or NCAM play an important role, and wherein stimulating of neurite outgrowth, cell survival, neural plasticity associated with memory and learning, and/or modulating cell motility is beneficial for treatment.

### FIELD OF INVENTION

The invention relates to new peptide compounds capable of stimulating neurite outgrowth, cell survival, neural plasticity associated with memory and learning, and modulating cell motility. The compounds of the invention are capable of binding and activating a neurotrophin receptor of the Trk family. The invention also relates to pharmaceutical compositions comprising the compounds and uses thereof for treatment of conditions wherein stimulating neurite outgrowth, neuronal cell survival, neural plasticity associated with memory and learning, and/or modulating cell motility is beneficial.

### BACKGROUND OF INVENTION

Neural cell adhesion molecules (CAMs) of the immunoglobulin superfamily nucleate and maintain groups of cells at key sites during early development and in the adult. In addition to their adhesive properties, CAMs homophilic and heterophilic interactions can affect intracellular signalling. Their ability to influence developmental events, including cell migration, proliferation, and differentiation may therefore result both from their adhesive as well as their signalling properties. However, accumulating evidence also indicates that cells may as well use the adhesive and signalling properties of CAMs in assistance of different physiologic processes separately, independently from each other.

The neural cell adhesion molecule, NCAM, was the first discovered neural CAM. Since the discovery NCAM has been intensively studied and now it is well characterised (Ronn et al. (2000) Int J Dev Neurosci 18:193-9). NCAM belongs to the immunoglobulin (Ig) superfamily. Its extracellular part consists of five Ig-like and two fibronectin type III (F3) modules. NCAM assists both the cell-cell and cell-substratum interactions. NCAM binds to various extracellular matrix components such as heparin/heparan sulfate, chondroitin sulfate proteoglycans, and different types of collagen. Cell-cell interactions are mostly assisted by the NCAM homophilic interaction. The different modules of NCAM have been shown to perform distinct functions. Thus, NCAM homophilic binding is believed now to depend on the first three Ig modules. The heparin binding sequence is localised to the Ig2 module. NCAM also binds to the neural cell adhesion molecule L1. This interaction is believed to take place between the fourth Ig module of NCAM and an oligomannosidic moiety expressed on L1. The two membrane-proximal F3 modules of NCAM have been shown involved in fibroblast growth factor receptor (FGFR) binding.

Many of the binding sites of NCAM involved in different interactions of the protein have been identified, and a number of short fragments of NCAM sequence comprising the sequences of different binding sites has been suggested to use as biologically active compounds in therapeutic applications (Berezin V., Bock E. (2004) J Mol. Neurosci. 22 (1-2):33-39). Many of these peptide fragments of NCAM are derived from homophilic NCAM binding sites and can therefore be used for stimulating cells expressing NCAM.

Among well-described heterophilic binding partners of NCAM is FGFR. Thus, NCAM has been regarded as a member of a new class of putative alternative ligands of FGFR, and recently there has been obtained evidence for a direct interaction between NCAM and the receptor (Kiselyov et al. (2003) Structure (Camb) 11:691-701). The identified NCAM fragment having the sequence EVYVVAENQQGKSKA (FGL peptide) involved in the direct interaction between NCAM and FGFR has recently been suggested as a new candidate compound for the treatment of a variety of pathologic disorders where the stimulation of activity of FGFR may play the key role (WO 03/016351). Interestingly, homophilic NCAM interaction leading to stimulation of neurite outgrowth has been shown in part also involves stimulation of FGFR (Soroka et al. (2002) J Biol. Chem. 277(27):24676-83).

Another class molecules described as NCAM ligands/receptors are heparan sulfate and chondroitin sulfate proteoglycans (HSPG and CSPG, respectively) (Cole et al. (1985). J. Cell Biol. 100(4):1192-9; Cole et al. (1986). Nature 320:445-7; Cole, G. J., Akeson, R. (1989) Neuron 2(2):1157-65). Multiple HSPGs and CSPGs can interact with NCAM in the brain influencing cell adhesion. Thus, NCAM expressed on the cell surface has the ability to bind to the HSPG agrin thereby mediating cell adhesion (Storms et al. (1996) Cell Adhes Commun. 3(6):497-509) and the brain-specific CSPG neurocan has been shown to bind to NCAM thereby inhibiting its homophilic interaction and subsequently neuronal adhesion (Retzler et al. (1996) J Biol. Chem. 271 (44):27304-10). When used as cell growth substrata, different synthetic peptides including amino acid sequences derived from a putative heparin binding domain of NCAM (HBD) identified in the Ig2 NCAM module have been shown to promote adhesion of NCAM expressing cells and are capable of moderate stimulation of neurite outgrowth (Cole, G. J., Akeson, R. (1989) Neuron 2(2):1157-65; Kallapur S. G., Akeson R. J. (1992) J Neurosci Res. 33(4): 538-48). Recently, a 14 amino acid sequence derived from NCAM HBD domain encompassing amino acid residues 152-165 of NCAM (SwissProt P13596) has been suggested as a fusion component of a fibrin 3D matrix gel for enhancing neurite outgrowth of neurons incorporated to this gel (US 2003/0119186). Another fragment of NCAM HBD comprising residues 149-166 was suggested as an inhibitor NCAM-mediated adhesion (U.S. Pat. No. 6,333,307), however biological activity of the latter fragment was not demonstrated.

HBD of NCAM comprising the sequence IWKHKGRDVILKKDVRFI contains two clusters of basic amino acids which have been demonstrated to be crucial for capacity of NCAM to bind heparin and also for NCAM homophilic adhesion. It has been shown that mutation of these basic amino acid residues affects biological activity of the domain (Kallapur S. G., Akeson R. J. (1992) J Neurosci Res. 33(4): 538-48; Cole, G. J., Akeson, R. (1989) Neuron 2(2):1157-65).

### SUMMARY OF INVENTION

According to the present invention:
1) a peptide fragment of the heparin binding domain (HBD) of NCAM corresponding to sequence comprising amino acid residues 154-167 of NCAM (SwissProt P13596), termed herein as heparin binding peptide (HBP), has a very high neuritogenic activity;
2) HBP mutants, wherein basic amino acid residues known to be important for biological activity of the peptide were substituted for other amino acid residues, are also capable of stimulating neurite outgrowth to the same extend as the non-mutated sequence of HBP;
3) neuritogenic activity of HBP and the HBP mutants is independent of i) NCAM-NCAM binding, ii) FGFR-NCAM binding and iii) HSPG- or CSPG-NCAM binding;
4) the peptide fragments of the invention are capable of modulating of cell motility;
5) the peptide fragments of the invention are active compounds both as immobile components of cell growth substrate and as soluble components of cell growth medium;
6) the peptide fragments of the invention comprise an amino acid motif, which is found in neurotrophins;
7) the peptide fragments of the invention are capable of binding to a neutrophin receptor of the Trk family.

Thus, one aspect of the present invention concerns a peptide, which is an isolated contiguous sequence of 6 to 13 amino acid residues, said sequence comprising the amino acid motif G-x^{a}-D/E/Q/T-V-x^{b}-V/L
wherein
x^{a} is any amino acid residue,
x^{b} is I, T, M or E.

Isolated peptide sequences comprising the above motif according to the invention are capable of i) binding to a neurotrophin receptor of the Trk family receptors; ii) stimulating neurite outgrowth; iii) modulating cell motility; iii) stimulating neural cell survival; iv) stimulating neural cell differentiation; and/or v) stimulating neural plasticity associated with learning and memory.

Accordingly, another aspect of the invention relates to using peptide sequences of the invention and/or compounds comprising said sequences for preparation of a medicament for treatment of a condition or disease wherein i) stimulating neurite outgrowth; ii) modulating cell motility; ii) stimulating neural cell survival; iii) stimulating neural cell differentiation; iv) stimulating neural plasticity; v) modulating activity of a neurotrophin receptor of the Trk family is part of said treatment.

Still, in another aspect a peptide sequence of the invention or a compound comprising said sequence may be used for the production of an antibody.

The invention further relates to compounds comprising peptide sequences of the invention and pharmaceutical compositions comprising said peptide sequences and/or said compounds. Pharmaceutical compositions comprising an antibody capable of recognising an epitope comprising the motif of the invention are also in the scope.

The invention also concerns a method of treatment of conditions wherein stimulating of neurite outgrowth, modulating of cell motility, stimulating of neural cell survival, stimulating of neural cell differentiation, stimulating neural plasticity, and/or modulating activity of a neurotrophin receptor of the Trk family receptors is beneficial, said method comprising a step of administering a peptide sequence of the invention, compound of the invention, antibody of the invention or a pharmaceutical composition comprising said peptide sequence, said compound or said antibody to an individual in need.

### DESCRIPTION OF DRAWINGS

FIG. 1 shows that NCAM expressing fibroblasts significantly promote neurite outgrowth of rat cerebella granular neurons (CGNs) as compared to control fibroblasts without NCAM expression, and HBP (SEQ ID NO: 1) in a dose-dependent manner increases neurite outgrowth of CGNs grown on fibroblasts expressing NCAM as well as on fibroblasts without NCAM expression. In contrast, the peptide M does not affect the neurite outgrowth response from CGNs grown on NCAM expressing fibroblasts, but it still stimulates neurite outgrowth of CGNs grown on fibroblasts without NCAM expression.

FIG. 2 demonstrates that the neuritogenic effect of HBP (SEQ ID NO: 1) on CGNs grown on monolayers of fibroblasts without NCAM expression is not blocked by treatment with SU5402, an inhibitor of the fibroblast growth factor receptor (FGFR), and NCAM mediated neurite outgrowth of CGNs grown on monolayers of NCAM expressing fibroblasts is only partially inhibited, however SU5402 blocks NCAM mediated neurite outgrowth in the absence of HBP.

FIG. 3 demonstrates the effect of HBP (SEQ ID NO: 1) and M peptide (SEQ ID NO: 2) on single CGNs in the presence or absence of heparinase III, the enzyme, which is known to specifically cleave heparan sulfate mainly into disaccharides.

FIG. 4 demonstrates a neuritogenic effect of peptides HBP (SEQ ID NO: 1), M (SEQ ID NO: 2), M1n (SEQ ID NO: 3), and M3n (SEQ ID NO: 4) in the presence and absence of heparinase III.

FIG. 5 demonstrates that treatment of both NCAM expressing fibroblasts and control fibroblasts without NCAM expression with HBP (SEQ ID NO: 1) and M peptide (SEQ ID NO: 2) results in a clear inhibition of cell motility as reflected by a pronounced decrease of the rate of diffusion (R), mean cell speed (Sτ), and locomotive index (LI) of cells.

FIG. 6 presents the results of analysis of different peptide fragments of NCAM HBD.

FIG. 7 demonstrates binding of HBP (SEQ ID NO: 1) (A) and recombinant Ig2 module of NCAM (B) to recombinant Trk B receptor.

### DETAILED DESCRIPTION OF THE INVENTION

### 1. Peptide Sequence

The first aspect of the present invention relates to an isolated contiguous peptide sequence consisting of 6 to 13 amino acid residues, comprising the amino acid motif

G-x^{a}-D/E/Q/T-V-x^{b}-V/L

wherein
x^{a} is any amino acid residue,
x^{b} is I, T, M or E.

In one embodiment x^{a} may be a basic amino acid residue. Some preferred embodiments concern x^{a} being K, other preferred embodiments concern x^{a} being R, and still other preferred embodiments concern x^{a} being H.

In another embodiment x^{a} may be L, and in still another embodiment x^{a} may be G.

Thus, the invention relates to a peptide sequence of at least 6 amino acids which are contiguous to each other and comprise the above motif, said peptide sequence being the isolated peptide sequence. This means that invention relates to a peptide sequence that exists as individual chemical entity and is used for the purposes of the invention as individual chemical entity. The invention does not relate to peptide sequences comprising the motif of the invention which 1) are isolated peptide sequences of 14 amino acid residues or more, e.g. bi-domain peptides of US 2003/0119186, such as the bi-domain peptide containing a peptide fragment of antithrombin III and fragment of NCAM HBD fused together in sequence LNEQVSPKHKGRDVILKKDVR; 2) are the integrated sequences of natural or recombinant polypeptides, such as for example polypeptides or fragments of the neural cell adhesion molecule (NCAM), nerve growth factor (NGF), neurotrophin-3 (NT-3), neurotriphin-4/5 (NT-4/5) or brain derived neurotrophic factor (BDNF) which are more then 14 amino acid long, 3) are integrated parts of any kind of 3D structure, e.g. fibrin or collagen gel.

More specifically, the invention concerns an isolated contiguous peptide sequence which may be defined by the formula (I)

x₀-x₁-x₂-x₃-x₄-x₅-x₆-x₇-x₈-x₉-x₁₀-x₉-x₉-x₁₀-x₁₁-x₁₂-x₁₃
Wherein
x₀ is K, R, N, A or a bond
x₁ is G,
x₂ is a basic or hydrophobic amino acid residue,
x₃ is E or D,
x₄ is V,
x₅ is a hydrophobic amino acid residue,
x₆ is V or L,
x₇ is any amino acid residue,
x₈ is any amino acid residue,
x₉ is E, D, K or Q,
x₁₀ is V or L,
x₁₁ is any amino acid residue,
x₁₂ is a hydrophobic amino acid residue or T,
x₁₃ is I, N, S, G, A or a bond.

The residue x₀ in some embodiments may be an amino acid residue selected from K, R, N, A. In other embodiments an isolated peptide sequence corresponding to the formula may not have the residue x₀. In this case the first residue of the sequence is x₁. x₀ may be a bond when the sequence is a part of a compound described below.

According to one preferred embodiment x₃ may be selected from Q, N or T. According to another preferred embodiment x₅ may be selected from T or E. Other preferred embodiments concerns the peptide sequences wherein x₇ and x₈ are independently selected from K, R, N, I, L, G, E or A. In some preferred embodiments x₇ is L, in other preferred embodiments x₈ may be G or E. Different preferred embodiments may concern the sequences wherein x₁₁ is selected from R, K, L, N or P and x₁₂ is a hydrophobic amino acid residue selected from F, V, I, T or A. x₁₃ may be selected from the amino acid residues I, N, S, G or A, or may be a bond when the sequence is a part of a compound described below. There are also some preferred embodiments when x₀ and/or x₁₃ is a bond.

Further, some preferred embodiments of the invention concern the sequences which may be defined by the formula (II)

x₀-G-x₂-D/E-V-I-L-x₇-x₈-x₉-V-x₁₁-x₁₂-x₁₃
wherein
x₀ is an amino acid residue selected from K, R, A or N,
x₂ is an amino acid residue selected from R or L,
x₇ is an amino acid residue selected from K, A, N or L,
x₈ is an amino acid residue selected from K, N or L,
x₉ is an amino acid residue selected from D or Q,
x₁₁ is an amino acid residue selected from R or L,
x₁₂ is an amino acid residue selected from V or F,
x₁₃ is an amino acid residue selected from I, L or V.

Other preferred embodiments of the invention may concern the sequences which may be defined by the formula (III)

x₀-G-x₂-x₃-V-x₅-V-L-G/E-x₉-V/L-x₁₀-x₁₁-x₁₂-x₁₃
wherein
x₀, x₂, x₃, x₅, x₉, x₁₀, as defined by the formula (I) above,
x₁₁ is N, K or P,
x₁₂ is I, T, A or V and
x₁₃ is S, A, N or G.

Different preferred embodiments may encompass the sequences wherein x₃ is Q and x₁₁ is selected from R, N, P or L.

Independent of different embodiments discussed above, peptide sequences corresponding to the formulas I, II and III, all, comprise the motif of the invention. The presence of a definite structure in a peptide sequence, for example a specific amino acid motif, is often a requirement for a common biological activity of a group of different peptide sequences. According to the present invention, the above described motif is required for biological activity of the sequences of the invention.

The invention identifies herein a group of peptide sequences comprising the above described motif which correspond to the formulas I, II or III, and demonstrates herein a common biological activity of these sequences, which is associated with said common structural feature (the motif) of the sequences. The group consists of the following sequences:

| | |
|---|---|
| KGRDVILKKDVRFI, | (SEQ ID NO: 1) |
| KGRDVILAKDVRVI, | (SEQ ID NO: 2) |
| KGRDVILNNDVRFI, | (SEQ ID NO: 3) |
| KGRDVILNNQVRFI. | (SEQ ID NO: 4) |
| AGRDVILNNDVRFI, | (SEQ ID NO: 5) |
| NGRDVILKKDVLFI, | (SEQ ID NO: 6) |
| NGLDVILIIDVRFI, | (SEQ ID NO: 7) |
| KGKEVMVLGEVNIN, | (SEQ ID NO: 8) |
| RGHQVTVLGEIKTG, | (SEQ ID NO: 9) |
| RGREVEVLGEVPAA | (SEQ ID NO: 10) |
| and | |
| SGGTVTVLEKVPVS. | (SEQ ID NO: 11) |

It is understood that the sequences of the above list represent a list of non-limited examples of sequences which comprise the motif of the invention and/or the motif of formula II or formula III and possess at least one common biological activity associated with the presence of these motifs in the structure of the peptide sequences. Biological activities associated with the described above structural features may be selected from but not limited to a capability of stimulating cell differentiation, memory and learning, neural cell survival, activating a neurotrophin receptor, or modulating cell motility.

An isolated peptide sequence comprising the motif G-x^{a}-D/E/Q/T-V-x^{b}-V/L, wherein x^{a} is any amino acid residue, x^{b} is I, T, M or E, according to the invention comprises 6 to 13 amino acid residues. However, it may be a sequence of 14 amino acid residues, if said sequence corresponds to formula I, II or III of above, such as for example sequences of SEQ ID NOs. 1-11. Invention also encompasses fragments of the sequences corresponding to the formulas I-III, in particular fragments of the sequences identified as SEQ ID NOs: 1-11, and also any peptides sequences of 6-13 amino acid residues long comprising said fragments. Thus, the fragments of the invention may be of 12, 11, 10, 9, 8, 7, or 6 amino acid residues long. A fragment that comprises any of the motifs of the formulas defined above is a preferred fragment of the invention. However, some embodiments may more preferably concern the fragments which comprise the motif G-x₂-D/E-V-I-L of formula II, wherein x₂ is R or L, whereas the other embodiments may more preferably concern the fragments which comprise the motif V-L-G/E-x₉-V/L of formula III, wherein x₉ is E, D, K or Q.

The invention also relates to variants and homologues of the sequences described above which possess at least some biological activity of said sequences. Preferably, such variants and homologues comprise an amino acid motif described above, namely
i) the motif of the invention: G-x^{a}-D/E/Q/T-V-x^{b}-V/L, wherein x^{a} is any amino acid residue, and x^{b} is I, T, M or E,
ii) the motif of formula II: G-x₂-D/E-V-I-L, wherein x₂ is R or L, or
iii) the motif of formula III: V-L-G/E-x₉-V/L, wherein x₉ is E, D, K or Q.

In the present application the standard one-letter code for amino acid residues as well as the standard three-letter code are applied. Abbreviations for amino acids are in accordance with the recommendations in the IUPAC-IUB Joint Commission on Biochemical Nomenclature Eur. J. Biochem, 1984, vol. 184, pp 9-37. Throughout the description and claims either the three letter code or the one letter code for natural amino acids are used. Where the L or D form has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms.

Where nothing is specified it is to be understood that the C-terminal amino acid of a peptide of the invention exists as the free carboxylic acid, this may also be specified as “—OH”. However, the C-terminal amino acid of a compound of the invention may be the amidated derivative, which is indicated as “—NH₂”. Where nothing else is stated the N-terminal amino acid of a polypeptide comprise a free amino-group, this may also be specified as “H—”.

Where nothing else is specified amino acid can be selected from any amino acid, whether naturally occurring or not, such as alfa amino acids, beta amino acids, and/or gamma amino acids. Accordingly, the group comprises but are not limited to: Ala, Val, Leu, Ile, Pro, Phe, Trp, Met, Gly, Ser, Thr, Cys, Tyr, Asn, Gln, Asp, Glu, Lys, Arg, His Aib, NaI, Sar, Orn, Lysine analogues, DAP, DAPA and 4Hyp.

Also, according to the invention modifications of the compounds/peptides may be performed, such as glycosylation and/or acetylation of the amino acids.

Basic amino acid residues are according to invention represented by the residues of amino acids Arg, Lys, and His, acidic amino acid residues—by the residues of amino acids Glu and Asp, hydrophobic amino acid residues—by the residues of amino acids Leu, Ile, Val, Phe, Trp, Tyr and Ala, and charged amino acid residues—by the residues of amino acids Arg, Lys, Glu and Asp.

As it was mentioned above the present invention relates to fragments, variant and homologues of the peptide sequences described above. In the present context
i) a fragment is a sequence which has at least 40%, more preferably at least 50%, more preferably at least 60%, more preferably at least 70%, more preferably at least 80%, more preferably at least 90%, more preferably at least 95% of the length of a sequence corresponding to a sequence defined by the formula I, II, or III or a sequence selected from the sequences of SEQ ID NOs: 1-11;
ii) a variant is an amino acid sequence having at least 60%, more preferably at least 70%, more preferably at least 80%, more preferably at least 90%, more preferably 95% homology to a sequence defined by formulas I, II or III, or to sequence selected from the sequences of SEQ ID NOs: 1-11, or is an amino acid sequence having at least 60%, more preferably at least 70%, more preferably at least 80%, more preferably at least 90%, more preferably 95% positive amino acid matches compared to a sequence defined by the formula I, II or II, or to a sequence of SEQ ID NOS: 1-10. A positive amino acid match is defined herein as an identity or similarity defined by physical and/or chemical properties of the amino acids having the same position in two compared sequences. Preferred positive amino acid matches of the present invention are K to R, E to D, L to M, Q to E, I to V, I to L, A to S, Y to W, K to Q, S to T, N to S and Q to R. The homology of one amino acid sequence with another amino acid is defined as a percentage of identical amino acids in the two collated sequences. The homology of the sequences may be calculated using well known algorithms such as BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, or BLOSUM 90;
iii) a homologue is an amino acid sequence which has less then 60% but more then 30%, such as 50-59%, for example 55%, such as 40-49%, for example 45%, such as 30-39%, for example 35% homology to a sequence defined by formula I, II or II, or to a sequence of SEQ ID NOs: 1-11.

It is presumed that the fragment, variant and homologue as described above remain at least some biological activity of the original sequence, for example a capability of stimulating neural plasticity, such as associated with neural cell differentiation and/or such as associated with memory and learning, stimulating of cell survival, such as inhibiting apoptosis, activating a neurotrophin receptor, such as activating Trk receptor, modulating cell motility, such as inhibiting cell motility

According to the invention a sequence as described above comprising the motif of the invention and/or the motif of formula II or formula III may derive from the sequence of the neural cell adhesion molecule (NCAM) of SwissProt Acc. No: P13596, for example from the sequence of the Ig2 module of NCAM. One example of such sequence may be sequence KGRDVILKKDVRFI identified herein as SEQ ID NO: 1. This sequence is a part of the heparin binding domain of the Ig module of NCAM. In another embodiment a sequence may derive from a neurtrophin, such as nerve growth factor (NGF), for example from the sequence of NGF polypeptide identified under SwissProt Acc. No: NP_{—}02497, neurotrophin-3 (NT-3), for example from the sequence of NT-3 polypeptide identified under of SwissProt Acc. No: NP_{—}002518, neurotrophin-4/5 (NT-4/5), for example from the sequence of NT-4/5 polypeptide identified under of SwissProt Acc. No: AAAV38176, or brain-derived neurotrophic factor (BDNF), for example from the sequence of BDNF polypeptide identified under of SwissProt Acc. No: NP_{—}733928. According to the invention sequence KGKEVMVLGEVNIN (SEQ ID NO: 8) is derived from the NGF polypeptide identified above, sequence RGHQVTVLGEIKTG (SEQ ID NO: 9) is derived from the NT-3 polypeptide identified above, sequence RGREVEVLGEVPAA (SEQ ID NO: 10) is derived from the NT-4/5 polypeptide identified above and sequence SGGTVTVLEKVPVS (SEQ ID NO: 11) is derived from the BDNF polypeptide identified above.

According to the present invention an isolated peptide sequence as described above may be formulated as a compound,

A compound may contain a single copy of an individual amino acid sequence selected from any of the described above, or it may contain two or more copies of such amino acid sequence. This means that compound of the invention may be formulated as a monomer of a peptide sequence, such as containing a single individual peptide sequence, or it may be formulated as a multimer of a peptide sequence, i.e containing two or more individual peptide sequences, wherein said individual peptide sequences may be represented by two or more copies of the same sequence or by two or more different individual peptide sequences. A multimer may also comprises a combination of the full-length sequence and one or more fragments thereof. In one embodiment a compound may contain two amino acid sequences, such compound is defined herein as dimer, in another embodiment a compound may contain more then two amino acid sequences, such for example three, four or more sequences. The present invention preferably relates to compounds containing two or four peptide sequences of the invention. However, compounds containing 3, 5, 6, 7, 8 or more sequences are also in the scope of the invention.

The peptide fragments formulated as dimers or multimers may have the identical amino acid sequences, or they may have different amino acid sequences. One example of such compound may be a compound containing SEQ ID NO: 1 and SEQ ID NO: 2. another example may be a compound containing SEQ ID NO: 1 and SEQ ID NO: 8. Any other combination of the sequences of the invention may be made depending on different embodiments of the invention. The sequences may be connected to each other via peptide bond, or connected to each other through a linker molecule or grouping. In another embodiment, compound may contain two or more identical copies of a sequence, such as for example two copies of a sequence selected from SEQ ID NO: 1, 2, 3, 4, 5, 6, 7, 8, 9, 10, and 11, wherein said two sequences may be connected to each other via a linker molecule or grouping. It is preferred a compound, wherein the sequences are connected via a linker grouping. One example of such linking grouping may be an achiral di-, tri- or tetracarboxylic acid. Suitable achiral di-, tri- or tetracarboxylic acids and a method of production such a compound (a ligand presentation assembly method (LPA)) are discussed in detail further in the specification of the invention. Another example of a possible linker may be the amino acid lysine. Individual peptide sequences may be attached to a core molecule such as lysine forming thereby a dendritic multimer (dendrimer) of an individual peptide sequence(s). Production of dendrimers is well known in the art (PCT/US90/02039, Lu et al., (1991) Mol. Immunol. 28:623-630; Defoort et al., (1992) Int J Pept Prot Res. 40:214-221; Drijfhout et al. (1991) Int J Pept Prot Res. 37:27-32), and dedrimers are at present widely used in research and in medical applications. It is a preferred embodiment of the invention to provide a dendrimeric compound comprising four individual amino acid sequences attached to the lysine core molecule. It is also preferred that at least one of the four individual amino acid sequences comprises an amino acid sequence of the formula defined above. It is even more preferred if the all four individual amino acid sequences of a dendrimeric compound individually comprise an amino acid sequence of the formula defined above.

Multimeric compounds of the invention, such as LPA-dimers or Lysin-dendrmers, are preferred compounds of the invention. However, other types of multimeric compounds comprising two or more individual sequences of the invention may be preferred depending on the embodiments.

Multimeric compounds, which comprise one or more copies of an individual sequence of the invention, or fragment, variant or homologue thereof, and another biologically active compound, for example another peptide sequence, are also in the scope of the invention. Among such compounds most preferable are those which comprise at least one peptide sequence of the invention and further comprise any other chemical entity which has a capability of stimulating a neuritrophin receptor of the Trk family, for example said chemical entity may be a peptide sequence derived from Trk receptor ligand or Trk receptor activator protein, wherein said sequence is different from the sequences of the invention.

### 2. Biological Activity

A peptide sequence of the invention and a compound comprising a sequence of the invention possess biological activity. The invention preferably relates to biological activity selected from a capability of stimulating neural plasticity associated with neural cell differentiation, such as for example stimulating neurite outgrowth, and/or neural plasticity associated with memory and learning, such as for example stimulating synaptic efficacy, capability of stimulating of cell survival, such as for example inhibiting apotosis, capability of activating a neurotrophin receptor, such as for example activating Trk receptor, and capability of modulating cell motility, such as for example inhibiting cell motility.

Thus, in one embodiment an isolated peptide sequence as described above is capable of binding to a neurotrophin receptor selected from Trk A, Trk B and Trk C. In one preferred embodiment, the receptor may be Trk A, in another preferred embodiment the receptor may be Trk B, in still another preferred embodiment the receptor may be Trk C.

The authors of the present invention has identified an amino acid motif which is present both in neurotrophins (NGF, NT-3, NT-4/5, BDNF) and NCAM and associated the presence of this motif with biological activity of peptide fragments of the present invention. The motif according to the invention is essential for binding of the peptide sequences of the invention to a Trk receptor, in particular to Trk B. Trk receptors are major receptors in the nervous system that drive differentiation of neural cells. Trk receptors are also important for promotion of neuronal survival and are involved in neural plasticity associated with learning and memory, in particular Trk B receptor. The capability of peptide sequences of the invention of binding and activating the receptors serves to modulating biological responses dependent on activity of the receptors. Accordingly, the invention provides a method of modulating activity of a neurotrophin receptor of the Trk family comprising using an isolated peptide sequence of the invention or a compound comprising said sequence. In preferred embodiment, the invention relates to a method for activating a neurotrophin receptor of the Trk family.

An isolated peptide sequence of the incention is capable of stimulating neuronal cell differentiation. The term “neuronal differentiation” is understood as both differentiation of neural precursor cells, or neural stem cells, and differentiation of neurons, such as maturation of differentiated neurons. An example of such differentiation may be neurite outgrowth from immature neurons, branching of neurites, neuron regeneration. In one preferred embodiment the invention may concern stimulating of differentiation of neural precursor/stem cells or immature neurons, in another preferred embodiment the invention may concern stimulating neurite outgrowth from mature neurons, for examples neurons which were traumatizes but survived. Thus, the invention also provides a method of stimulating of neuronal cell differentiation comprising using a peptide sequence of the invention or a compound comprising said sequence.

One most preferred embodiment of the invention concerns the activity of the peptide sequences in connection with learning and memory. In particular, one embodiment of the invention the peptide sequences may stimulate spine formation, in another embodiment the sequences may promote synaptic efficacy. Thus, the invention further provides a method for stimulating memory and/or learning comprising using a peptide sequence of the invention and/or compound comprising said sequence. The invention relates to both short-term memory and long-term memory.

A peptide sequence of the invention may also stimulate neuronal cell survival. The invention concerns the capability of stimulating neuronal cell survival both due trauma and degenerative disease. Accordingly, the invention provides a method for stimulating cell survival, preferably neuronal cell survival by using a peptide sequence of the invention and/or compound comprising said sequence.

In still another embodiment a peptide sequence of the invention may modulate cell motility. The term “modulating cell motility” includes both stimulating and inhibiting cell motility. The activity of a peptide sequence which results in inhibiting cell motility is preferred. Thus, a method for modulating cell motility, in particular inhibiting cell motility, by using a peptide sequence of the invention or a compound comprising said sequence is also among the methods for modulating cellular and physiological processes provided by the invention.

The peptide sequences of the invention and compounds comprising thereof are biologically active both as soluble/mobile substances of cell growth media and immobile substances of cell growth substrate. In some embodiments it may be preferred to use a peptide substance or a compound comprising thereof as cell substrate. However, soluble peptide sequences or compounds comprising thereof are most preferred.

Non-limited examples of biological activity of the peptide sequences of the invention and compounds comprising thereof are described in the application (see below and Examples 2, 3 and 5).

### Stimulation of Neurite Outgrowth

Substances with the potential to promote neurite outgrowth as well as stimulate regeneration and/or differentiation of neuronal cells, such as certain endogenous trophic factors; are prime targets in the search for compounds that facilitate for example neuronal regeneration and other forms of neuronal plasticity. To evaluate the potential of the present compound, the ability to stimulate the neurite outgrowth related signalling, interfere with cell adhesion, stimulate neurite outgrowth, regeneration of nerves, may be investigated. Compounds of the present invention are shown to promote neurite outgrowth and are therefore considered to be good promoters of regeneration of neuronal connections, and thereby of functional recovery after damages as well as promoters of neuronal function in other conditions where such effect is required.

In the present context “differentiation” is related to the processes of maturation of neurons and extension of neurites, which take place after the last cell division of said neurons. The compounds of the present invention may be capable of stopping neural cell division and initiating maturation said cells, such as initiating extension of neurites. Otherwise, “differentiation” is related to initiation of the process of genetic, biochemical, morphological and physiological transformation of neuronal progenitor cells, immature neural cells or embryonic stem cells leading to formation of cells having functional characteristics of normal neuronal cell as such characteristics are defined in the art. The invention defines “immature neural cell” as a cell that has at least one feature of neural cell accepted in the art as a feature characteristic for the neural cell.

According to the present invention a compound comprising at least one of the above peptide sequences is capable of stimulating neurite outgrowth. The invention concerns the neurite outgrowth improvement/stimulation such as about 75% improvement/stimulation above the value of neurite outgrowth of control/non-stimulated cells, for example 50%, such as about 150%, for example 100%, such as about 250, for example 200%, such as about 350%, for example 300%, such as about 450%, for example 400%, such as about 500%.

Estimation of capability of a candidate compound to stimulate neurite outgrowth may be done by using any known method or assay for estimation of neurite outgrowth, such as for example the one described in Example 2 of the present application.

According to the invention a compound has neuritogenic activity both as an insoluble immobile component of cell growth substrate and as a soluble component of cell growth media. In the present context “immobile” means that the compound is bound/attached to a substance which is insoluble in water or a water solution and thereby it becomes insoluble in such solution as well. For medical applications both insoluble and soluble compounds are considered by the application, however soluble compounds are preferred. Under “soluble compound” is understood a compound, which is soluble in water or a water solution.

### Inhibition of Cell Motility

Cell migration is required during development of the nervous system, wound healing and tumor invasion. The correct formation and normal function of the nervous system both require that the majority of neurons migrate throughout the developing nervous system from their sites of origin to their final positions.

Some types of cells maintain a capacity to move also in a mature organism, whereas the other types lose it. In some extreme conditions such as in disease or trauma, a capability of a cell to move may define the onset of rescue or death from the disease, such as wound healing or cancer cells invasion and metastases. Therefore, sub-stances with the potential to modulate cell motility, such as certain endogenous trophic factors, are prime targets in the search for compounds that for example facilitate the recovery from trauma, prevent the dissemination of cancer cells or inhibit the spreading of inflammation. To evaluate the potential of the present compound, the ability of modulating of signalling related to cell motility, interfering with cell adhesion, stimulating or inhibiting cell motility, may be investigated. Compounds of the present invention are capable of inhibiting of cell motility and are, therefore, considered to be good candidate compounds for inhibiting for example invasion and dissemination of cancer cells as well as inhibiting of any type cell invasion in conditions when such inhibition is required.

According to the present invention a compound comprising at least one of the above sequences is capable of inhibiting of cell motility. The invention concerns the inhibition of cell motility, which is estimated to be of about 75% when compared to control cells motility, for example about 50%, such as about 150%, for example about 100%, such as about 250%, for example about 200%, such as about 350%, for example about 300%, such as about 450%, for example about 400%, such as for example about 500%. The term “motility” is defined herein as displacement of a cell from a place where it was to another place in a certain period of time, and in the present application cell motility is estimated as the Euclidean distance between two points corresponding to the initial and final positions of the cell. When considering quantification of cell motility and the inhibitory potential of compounds, such as the above mentioned “value” of inhibition or motility, the present application relates to the “values” defined such parameters as the rate of diffusion (R), mean-cell speed (Sτ) and locomotive index (LI) of cells. The later parameters are commonly used in the art for quantification of cell motility and described for example by Walmod et al. (2001) Methods Mol. Biol. 161:59-83, and featured below.

Analysis of cell motility may be done by using any available methods and assays developed in the art for the purpose. It may be done for example as described in the present application in Example 3.

### 3. Production of Peptide Sequences

Compounds of the present invention are preferably produced synthetically. However, recombinant production of the compounds is also contemplated.

### Recombinant Production

Thus, in one embodiment the peptide sequences of the invention may be produced by use of recombinant DNA technologies.

The DNA sequence encoding a peptide or the corresponding full-length protein the peptide originates from may be prepared synthetically by established standard methods, e.g. the phosphoamidine method described by Beaucage and Caruthers, 1981, Tetrahedron Lett. 22:1859-1869, or the method described by Matthes et al., 1984, EMBO J. 3:801-805. According to the phosphoamidine method, oligonucleotides are synthesised, e.g. in an automatic DNA synthesiser, purified, annealed, ligated and cloned in suitable vectors.

The DNA sequence encoding a peptide may also be prepared by fragmentation of the DNA sequences encoding the corresponding full-length protein of peptide origin, using DNAase I according to a standard protocol (Sambrook et al., Molecular cloning: A Laboratory manual. 2 rd ed., CSHL Press, Cold Spring Harbor, N.Y., 1989). The present invention relates to full-length proteins selected from the groups of proteins identified above. The DNA encoding the full-length proteins of the invention may alternatively be fragmented using specific restriction endonucleases. The fragments of DNA are further purified using standard procedures described in Sambrook et al., Molecular cloning: A Laboratory manual. 2 rd ed., CSHL Press, Cold Spring Harbor, N.Y., 1989.

The DNA sequence encoding a full-length protein may also be of genomic or cDNA origin, for instance obtained by preparing a genomic or cDNA library and screening for DNA sequences coding for all or part of the full-length protein by hybridisation using synthetic oligonucleotide probes in accordance with standard techniques (cf. Sambrook et al., Molecular Cloning: A Laboratory Manual, 2nd Ed., Cold Spring Harbor, 1989). The DNA sequence may also be prepared by polymerase chain reaction using specific primers, for instance as described in U.S. Pat. No. 4,683,202 or Saiki et al., 1988, Science 239:487-491.

The DNA sequence is then inserted into a recombinant expression vector, which may be any vector, which may conveniently be subjected to recombinant DNA procedures. The choice of vector will often depend on the host cell into which it is to be introduced. Thus, the vector may be an autonomously replicating vector, i.e. a vector that exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

In the vector, the DNA sequence encoding a peptide or a full-length protein should be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing the transcription of the coding DNA sequence in mammalian cells are the SV 40 promoter (Subramani et al., 1981, Mol. Cell. Biol. 1:854-864), the MT-1 (metallothionein gene) promoter (Palmiter et al., 1983, Science 222: 809-814) or the adenovirus 2 major late promoter. A suitable promoter for use in insect cells is the polyhedrin promoter (Vasuvedan et al., 1992, FEBS Lett. 311:7-11). Suitable promoters for use in yeast host cells include promoters from yeast glycolytic genes (Hitzeman et al., 1980, J. Biol. Chem. 255:12073-12080; Alber and Kawasaki, 1982, J. Mol. Appl. Gen. 1: 419-434) or alcohol dehydrogenase genes (Young et al., 1982, in Genetic Engineering of Microorganisms for Chemicals, Hollaender et al, eds., Plenum Press, New York), or the TPI1 (U.S. Pat. No. 4,599,311) or ADH2-4-c (Russell et al., 1983, Nature 304:652-654) promoters. Suitable promoters for use in filamentous fungus host cells are, for instance, the ADH3 promoter (McKnight et al., 1985, EMBO J. 4:2093-2099) or the tpiA promoter.

The coding DNA sequence may also be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) promoters. The vector may further comprise elements such as polyadenylation signals (e.g. from SV 40 or the adenovirus 5 E1b region), transcriptional enhancer sequences (e.g. the SV 40 enhancer) and translational enhancer sequences (e.g. the ones encoding adenovirus VA RNAs).

The recombinant expression vector may further comprise a DNA sequence enabling the vector to replicate in the host cell in question. An example of such a sequence (when the host cell is a mammalian cell) is the SV 40 origin of replication. The vector may also comprise a selectable marker, e.g. a gene the product of which complements a defect in the host cell, such as the gene coding for dihydrofolate reductase (DHFR) or one which confers resistance to a drug, e.g. neomycin, hydromycin or methotrexate.

The procedures used to ligate the DNA sequences coding the peptides or full-length proteins, the promoter and the terminator, respectively, and to insert them into suitable vectors containing the information necessary for replication, are well known to persons skilled in the art (cf., for instance, Sambrook et al., op.cit.).

To obtain recombinant peptides of the invention the coding DNA sequences may be usefully fused with a second peptide coding sequence and a protease cleavage site coding sequence, giving a DNA construct encoding the fusion protein, wherein the protease cleavage site coding sequence positioned between the HBP fragment and second peptide coding DNA, inserted into a recombinant expression vector, and expressed in recombinant host cells. In one embodiment, said second peptide selected from, but not limited by the group comprising glutathion-S-reductase, calf thymosin, bacterial thioredoxin or human ubiquitin natural or synthetic variants, or peptides thereof. In another embodiment, a peptide sequence comprising a protease cleavage site may be the Factor Xa, with the amino acid sequence IEGR, enterokinase, with the amino acid sequence DDDDK, thrombin, with the amino acid sequence LVPR/GS, or Acharombacter lyticus, with the amino acid sequence XKX, cleavage site.

The host cell into which the expression vector is introduced may be any cell which is capable of expression of the peptides or full-length proteins, and is preferably a eukaryotic cell, such as invertebrate (insect) cells or vertebrate cells, e.g. Xenopus laevis oocytes or mammalian cells, in particular insect and mammalian cells. Examples of suitable mammalian cell lines are the HEK293 (ATCC CRL-1573), COS (ATCC CRL-1650), BHK (ATCC CRL-1632, ATCC CCL-10) or CHO (ATCC CCL-61) cell lines. Methods of transfecting mammalian cells and expressing DNA sequences introduced in the cells are described in e.g. Kaufman and Sharp, J. Mol. Biol. 159, 1982, pp. 601-621; Southern and Berg, 1982, J. Mol. Appl. Genet. 1:327-341; Loyter et al., 1982, Proc. Natl. Acad. Sci. USA 79: 422-426; Wigler et al., 1978, Cell 14:725; Corsaro and Pearson, 1981, in Somatic Cell Genetics 7, p. 603; Graham and van der Eb, 1973, Virol. 52:456; and Neumann et al., 1982, EMBO J. 1:841-845.

Alternatively, fungal cells (including yeast cells) may be used as host cells. Examples of suitable yeast cells include cells of Saccharomyces spp. or Schizosaccharomyces spp., in particular strains of Saccharomyces cerevisiae. Examples of other fungal cells are cells of filamentous fungi, e.g. Aspergillus spp. or Neurospora spp., in particular strains of Aspergillus oryzae or Aspergillus niger. The use of Aspergillus spp. for the expression of proteins is described in, e.g., EP 238 023.

The medium used to culture the cells may be any conventional medium suitable for growing mammalian cells, such as a serum-containing or serum-free medium containing appropriate supplements, or a suitable medium for growing insect, yeast or fungal cells. Suitable media are available from commercial suppliers or may be pre-pared according to published recipes (e.g. in catalogues of the American Type Culture Collection).

The peptides or full-length proteins recombinantly produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. HPLC, ion exchange chromatography, affinity chromatography, or the like.

### Synthetic Production of Individual Peptide Sequences

The methods for synthetic production of peptides are well known in the art. Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999.

Peptides may for example be synthesised by using Fmoc chemistry and with Acm-protected cysteins. After purification by reversed phase HPLC, peptides may be further processed to obtain for example cyclic or C- or N-terminal modified isoforms. The methods for cyclization and terminal modification are well-known in the art and described in detail in the above-cited manuals.

In a preferred embodiment the individual peptide sequences of the invention are produced synthetically, in particular, by the Sequence Assisted Peptide Synthesis (SAPS) method described in the above manuals.

Otherwise, the synthesis of an individual peptide sequence of the invention may be ordered and purchased from a commercial manufacturer, such as for example Sigma-Genosys (USA).

### Production of Multimers of Individual Peptide Sequences LPA Method

The LPA method is disclosed in WO 00/18791. The method essentially comprises the following steps:
(a) providing by solid phase synthesis or fragment coupling peptide fragment(s) comprising the desired sequencers), the peptide fragment(s) being attached to a solid phase;
(b) if necessary, deprotecting any N-terminal amino groups whole the peptide fragment(s) are still attached to a solid phase,
(c) reacting the peptide fragment(s) having unprotected N-terminal groups with an achiral di-, tri-, or tetracarboxylic acid so as to provide a construct having a ring structure, and
(d) cleaving the construct from the solid phase so as to provide an LPA comprising the peptide fragment(s) having free C-terminal groups.

In the above method, prior step (d) the following steps may be performed:

(c1) if present, deprotecting any N-protected groups originating from the carboxylic acid used in step (c),
(c2) continuing the solid phase synthesis or fragment coupling so as to provide peptide fragment(s) comprising desired sequence(s) having at least one N-protected N-terminal amino acid group and/or attaching chemical moieties, and
(c3) deprotecting, if present, any N-terminal amino groups (prior or after step (d)).

The method provides i.a. LPAs presenting desired sequences of the invention with N to C orientation (step (c)). And also simultaneously sequences with C to n orientation (step (c2))

Thus, to obtain a compound of the invention two peptide chains attached to a solid phase are to be assembled by means of achiral di-, tri- or tetracarboxylic acids. Suitable achiral di-, tri- or tetracarboxylic acids to be used in the present method have the general formula

X[(A)nCOOH][(B)mCOOH]

wherein n and m independently are an integer of from 1 to 20, X is HN, A and B independently are a substituted or unsubstituted C₁₋₁₀ alkyl, a substituted or unsubstituted C₂₋₁₀ alkenyl, a substituted or unsubstituted cyclic moiety, a substituted or unsubstituted heterocyclic moiety, a substituted or unsubstituted aromatic moiety, or A and B together form a substituted or unsubstituted cyclic moiety, substituted or un-substituted heterocyclic moiety, substituted or unsubstituted aromatic moiety.

In another embodiment suitable achiral di-, tri- or tetracarboxylic acids to be used in the present method have the general formula

X[(A)nCOOH][(B)mCOOH]

wherein n and m are 0 or an integer of from 1 to 20, X is H₂N(CR₂)pCR, or RHN(CR₂)pCR, wherein p is 0 or integer of from 1 to 20, wherein each R is H, a substituted or unsubstituted C₁₋₁₀ alkyl, a substituted or unsubstituted C₂₋₁₀ alkenyl, a substituted or unsubstituted cyclic moiety, a substituted or unsubstituted heterocyclic moiety, a substituted or unsubstituted aromatic moiety, or A and B together form a substituted or unsubstituted cyclic moiety, substituted or unsubstituted heterocyclic moiety, substituted or unsubstituted aromatic moiety.

In still another embodiment suitable achiral di-, tri- or tetracarboxylic acids to be used in the present method have the general formula

X[(A)nCOOH][(B)mCOOH]

wherein n and m are 0 or an integer of from 1 to 20, X is HO(CR₂)pCR, HS(CR₂)pCR, halogen-(CR₂)pCR, HOOC(CR₂)pCR, ROOC(CR₂)pCR, HCO(CR₂)pCR, RCO(CR₂)pCR, or [HOOC(A)n][HOOC(B)m]CR(CR₂)pCR, wherein p is 0 or integer of from 1 to 20, each R independently is H or a substituted or un-substituted C₁₋₁₀ alkyl, a substituted or unsubstituted C₂₋₁₀ alkenyl, a substituted or unsubstituted cyclic moiety, a substituted or unsubstituted heterocyclic moiety, a substituted or unsubstituted aromatic moiety, or A and B together form a substituted or unsubstituted cyclic moiety, substituted or unsubstituted heterocyclic moiety, substituted or unsubstituted aromatic moiety.

In yet another embodiment suitable achiral di-, tri- or tetracarboxylic acids to be used in the present method have the general formula

X[(A)nCOOH][(B)mCOOH]

Wherein n and m are 0 or an integer of from 1 to 20, X is H₂N(CR₂)p, RHN(CR₂)p, HO(CR₂)p, HS(CR₂)p, halogen-(CR₂)p, HOOC(CR₂)p, ROOC(CR₂)p, HCO(CR₂)p, RCO(CR₂)p, or [HOOC(A)n][HOOC(B)m](CR₂)p, wherein p is 0 or integer of from 1 to 20, each R independently is H or a substituted or unsubstituted C₁₋₁₀ alkyl, a substituted or unsubstituted C₂₋₁₀ alkenyl, a substituted or unsubstituted cyclic moiety, a substituted or unsubstituted heterocyclic moiety, a substituted or unsubstituted aromatic moiety, or A and B together form a substituted or unsubstituted cyclic moiety, substituted or unsubstituted heterocyclic moiety, substituted or unsubstituted aromatic moiety.

Under the term C₁₋₁₀ alkyl is meant straight or branched chain alkyl groups having 1-10 carbon atoms, e.g. methyl, ethyl, isopropyl, butyl, and tertbutyl.

Under the term C₂₋₁₀ alkenyl is meant straight or branched chain alkenyl groups having 2-10 carbon atoms, e.g. ethynyl, propenyl, isopropenyl, butenyl, and tert-butenyl.

Under the term cyclic moiety is meant cyclohexan, and cyclopentane.

Under the term aromatic moiety is meant phenyl.

The wording “A and B forms a cyclic, heterocyclic or aromatic moiety” denotes cyclohexan, piperidine, benzene, and pyridine.

By reaction with a carboxylic acid, a construct of the type

X(CO-sequence)₂-solid phase, wherein X as defined above,
is obtained.

By the term “sequence” is in the present content meant a peptide comprising naturally occurring and/or non-naturally occurring amino acids, a PNA-sequence, or peptidomimetic. By “naturally occurring amino acids” is meant L- and D-forms of the 20 acids found in nature. Non-naturally occurring amino acids are e.g. modified naturally occurring amino acids. The term sequence is further intended to comprise one or more of such sequences. Examples of suitable peptidomimetics are described in Marshall G. R., (1993) Tetrahedron, 49:3547-3558. The term “chemical moieties” denotes an entity enhancing the solubility or biological activity of the LPA, and entity for directing the LPA to its target or a marker. Preferred embodiments for the sequences are described above.

The group X permits directly or indirectly continued stepwise synthesis or a fragment coupling of the same sequence, or of one or more different sequences and/or moieties. Orientation of peptide fragments (N to C or C to N) in LPA is defined as desired. In one embodiment the present invention features LPAs with N to C orientation, in another embodiment it concerns the compounds with simultaneous N to C and C to N presentation of the sequences, and in yet another embodiment the sequences have C to N orientation.

In the case where X comprises a temporally protected amino function, synthesis or coupling can be carried out directly after protection. Suitable activation of all carboxyl-containing groups providing effective formation of the ring system (on step (c), see above) can be ensured using half-equivalent carboxy acid. In case of tri- or tetracarboxylic acids the activated carboxy group may further be derivatised with a diamine such as ethylenediamine or an amine suitably functionalised for further reactions such as mercapto-, an oxy-, an oxo or carboxyl group. In the case of diamine, peptide synthesis or fragment coupling can be continued directly according to the desired sequence or chemical moiety. In a preferred embodiment, the Fmoc-protection strategy is used, but any amino protection group may be used depending on the synthesis or coupling strategy. Examples are the Boc-protection group strategy.

Since the continued stepwise synthesis or fragment coupling is performed with one or in case of a bifunctional chemical moiety such lysine with two amino acid groups, it has surprisingly been found that a much better result can be obtained as compared to conventional tetrameric lysine dendimers obtained by the MAP synthesis. Furthermore, optimal peptide synthesis procedures or coupling procedures can be used for the single chains attached to the solid phase, and their homogeneity can be verified prior to forming the LPA. Cleavage from the solid phase and simultaneous side-chain deprotection can be performed by standard peptide synthesis procedures (described above). A final product may thus be obtained having optimal and well-defined composition. Purification by standard chromatography methods such as HPLC or gel filtration can easily be performed, if desired or needed.

Favourable di-, tri- and tetracarboxilyc acids for providing the ring structure may be selected from imino diacetic acid, 2-amino malonic acid, 3-amino glutaric acid, 3-methylamino glutaric acid, 3-chloro glutamic acid, 3-methoxy-carbonyl glutaric acid, 3-acetyl glutaruc acid, glutaric acid, tricarballylic acid, 3,4-bis-carboxymethyl adipic acid, 4-(2-carboxyethyl)-pimelic acid, (3,5-bis-carboxymethyl-phenyl)-acetic acid, 3,4-bis-carboxymethyl-adipic acid, benzene-1,2,4,5-tetra carboxylic acid, 4-(3-carboxy-allylamino)-but-2-enoic acid, 4,4-imino-dibenzoic acid, 1,4-dihydropyridine-3,5-dicarboxylic acid, 5-amino isophthalic acid, 2-chloro malonic acid, 3-hydroxy glutaric acid, and benzene-1,3,5-tricarboxylic acid.

Fragment coupling (fragment coupling or fragment condensation) may be performed according to standard procedures, e.g. as described in Peptide Synthesis protocols, Methods in Molecular Biology Vol. 35, Chapter 15, 303-316, Nyfeler R, Pennington M W and Dunne B M Eds., Humana Press, 1994. Accordingly, fragments may be synthesised on a solid phase, cleaved from the solid phase with full preservation of protecting groups, purified and characterised as described above. Suitable fragments may also be obtained by other techniques described above.

It is a preferred embodiment of the invention to use the above LPA method for the production of a compound of the invention.

### 4. Antibody

It is an objective of the present invention to provide an antibody, antigen binding fragment or recombinant protein thereof capable of recognizing and selectively binding to an epitope comprising or comprised by a sequence corresponding to formula I of the invention, or fragment, variant or homologue of said sequence, in a preferred embodiment an epitope comprising a motif of the invention. In one preferred embodiment the antibody is an antibody that recognizes and binds to an epitope comprising the motif G-x^{a}-D/E/Q/T-V-x^{b}V/L, wherein x^{a} is any amino acid residue, x^{b} is i, T, M or E. In another preferred embodiment the antibody is an antibody that recognizes and bind to an epitope comprising the motif G-x₂-D/E-V-I-L, wherein x₂ is R or L. In still another preferred embodiment the antibody is an antibody that recognizes and binds to an epitope comprising the motif V-L-G/E-x₉V/L, wherein x₉ is E, D, K or Q. In other preferred embodiments the antibody may be selected from antibodies that recognizes and binds to an epitope comprising or comprised by a sequence selected from any of the sequences of SEQ ID NOs: 1-11, or a fragment, or variant or homologue of said sequences.

Preferably, the epitope is located on NCAM, NGF, NT-3 or NT-4/5 polypeptide.

By the term “epitope” is meant the specific group of atoms (on an antigen molecule) that is recognized by (that antigen's) antibodies (thereby causing an immune response). The term “epitope” is the equivalent to the term “antigenic determinant”. The epitope may comprise 3 or more amino acid residues, such as for example 4, 5, 6, 7, 8 amino acid residues, located in close proximity, such as within a contiguous amino acid sequence, or located in distant parts of the amino acid sequence of an antigen, but due to protein folding have been approached to each other.

Antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Novotny J, & Haber E. Proc Natl Acad Sci USA. 82(14):4592-6, 1985).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely a adopting a β-sheet configuration, connected by three CDRs, which form loops connecting, and in some cases forming part of, the I-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific antigen. In preferred embodiments, in the context of both the therapeutic and screening methods described below, an antibody or fragment thereof is used that is immunospecific for an antigen or epitope of the invention.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from anti-body fragments. As used herein, “functional fragment” with respect to antibodies, refers to Fv, F(ab) and F(ab′)₂ fragments.

The term “antibody fragment” is used herein interchangeably with the term “antigen binding fragment”.

Antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more. In general, an antibody fragment of the invention can have any upper size limit so long as it is has similar or immunological properties relative to antibody that binds with specificity to an epitope comprising a peptide sequence selected from any of the sequences identified herein as SEQ ID NOs: 1-11, or a fragment of said sequences. Thus, in context of the present invention the term “antibody fragment” is identical to term “antigen binding fragment”.

Antibody fragments retain some ability to selectively bind with its antigen or receptor. Some types of antibody fragments are defined as follows:
(1) Fab is the fragment that contains a monovalent antigen-binding fragment of an antibody molecule. A Fab fragment can be produced by digestion of whole antibody with the enzyme papain to yield an intact light chain and a portion of one heavy chain.
(2) Fab′ is the fragment of an antibody molecule can be obtained by treating whole antibody with pepsin, followed by reduction, to yield an intact light chain and a portion of the heavy chain. Two Fab′ fragments are obtained per anti-body molecule.

Fab′ fragments differ from Fab fragments by the addition of a few residues at the carboxyl terminus of the heavy chain CH1 domain including one or more cysteines from the antibody hinge region.
(3) (Fab′)₂ is the fragment of an antibody that can be obtained by treating whole antibody with the enzyme pepsin without subsequent reduction.
(4) F(ab′)₂ is a dimer of two Fab′ fragments held together by two disulfide bonds.

Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.
(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies 113: 269-315 Rosenburg and Moore eds. Springer-Verlag, N.Y., 1994.

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad. Sci. USA 90: 6444-6448 (1993).

The invention contemplate both polygonal and monoclonal antibody, antigen binding fragments and recombinant proteins thereof which are capable of binding an epitope according to the invention.

The preparation of polyclonal antibodies is well-known to those skilled in the art. See, for example, Green et al 1992. Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1, which are hereby incorporated by reference.

The preparation of monoclonal antibodies likewise is conventional. See, for example, Kohler & Milstein, Nature, 256:495-7 (1975); Coligan, et al., sections 2.5.1-2.6.7; and Harlow, et al., in: Antibodies: A Laboratory Manual, page 726, Cold Spring Harbor Pub. (1988), Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG). In: Methods in Molecular Biology, 1992, 10:79-104, Humana Press, NY.

Methods of in vitro and in vivo manipulation of monoclonal antibodies are well known to those skilled in the art. For example, the monoclonal antibodies to be used in accordance with the present invention may be made by the hybridoma method first described by Kohler and Milstein, 1975, Nature 256, 495-7, or may be made by recombinant methods, e.g., as described in U.S. Pat. No. 4,816,567. The monoclonal antibodies for use with the present invention may also be isolated from phage antibody libraries using the techniques described in Clackson et al., 1991, Nature 352: 624-628, as well as in Marks et al., 1991, J Mol Biol 222: 581-597. Another method involves humanizing a monoclonal antibody by recombinant means to generate anti-bodies containing human specific and recognizable sequences. See, for review, Holmes, et al., 1997, J Immunol 158:2192-2201 and Vaswani, et al., 1998, Annals Allergy, Asthma & Immunol 81:105-115.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in anti-bodies derived from another species or belonging to another antibody class or subclass, as well as fragments of such antibodies, so long as they exhibit the desired biological activity (U.S. Pat. No. 4,816,567); Morrison et al., 1984, Proc Natl Acad Sci 81: 6851-6855.

Methods of making antibody fragments are also known in the art (see for example, Harlow and Lane, Antibodies: A Laboratory Manual, Cold Spring Harbor Laboratory, NY, 1988, incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, in U.S. Pat. No. 4,036,945 and U.S. Pat. No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., 1991, In: Methods: A Companion to Methods in Enzymology, 2:97; Bird et al., 1988, Science 242:423-426; U.S. Pat. No. 4,946,778; and Pack, et al., 1993, BioTechnology 11:1271-77.

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain a minimal sequence derived from non-human immunoglobulin, such as the eitope recognising sequence. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity. Humanized antibody(es) containing a minimal sequence(s) of antibody(es) of the invention, such as a sequence(s) recognising an epitope(s) described herein, is one of the preferred embodiments of the invention.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., 1986, Nature 321, 522-525; Reichmann et al., 1988, Nature 332, 323-329; Presta, 1992, Curr Op Struct Biol 2:593-596; Holmes et al., 1997, J Immunol 158:2192-2201 and Vaswani, et al., 1998, Annals Allergy, Asthma & Immunol 81:105-115.

The generation of antibodies may be achieved by any standard methods in the art for producing polyclonal and monoclonal antibodies using natural or recombinant fragments of NCAM, NGF, NT3 or NT4/5 which comprise a the motif of the invention, such as for example the sequences identified as SEQ ID NOs: 1-11, as an antigen. Such antibodies may be also generated using variants, homologues or fragments of peptide sequences of SEQ ID NOs: 1-11, or any other immunogenic peptide sequences or immunogenic fragments thereof, which meet the following criteria:

(i) being a contiguous amino acid sequence of at least 6 amino acids, and
(ii) comprising at least one amino acid motif discussed above.

The antibodies may also be produced in vivo by the individual to be treated, for example, by administering an immunogenic fragment according to the invention to said individual. Accordingly, the present invention further relates to a vaccine comprising an immunogenic fragment described above.

The application also relates to a method for producing an antibody of the invention said method comprising a step of providing of an immunogenic fragment described above.

The invention relates both to 1) an antibody, which is capable of modulating, such as enhancing or attenuating, biological function of human NCAM and/or a neurotrophin and/or Trk receptor, in particular a function related to cell growth, differentiation and survival, neural plasticity associated with learning and memory, and/or cell motility, 2) an antibody, which can recognise and specifically bind to NCAM and/or to a Trk receptor ligand, such as NGF, NT-3 or NT-4/5, and modulate the function of NCAM and/or Trk receptor ligand; and 3) an antibody, which can recognise and specifically bind to NCAM and/or a Trk receptor ligand, such as NGF, NT-3 or NT-4/5, without modulating biological activity thereof. It is preferred that such antibody is produced by using an immunogenic peptide sequence described above.

The invention relates to use of the described above antibodies for 1) therapeutic applications involving the modulation of activity of neurotrophins, NCAM and/or Trk receptors; 2) for modulating cellular and physiological processes including cell differentiation, survival and motility, and neural plasticity associated with learning and memory 3) detecting and/or monitoring neurotrophins, NCAM and/or Trk receptors in vitro and/or in vivo for diagnostic purposes, 4) research purposes.

In one embodiment the invention relates to a pharmaceutical composition comprising an antibody described above.

### 5. Medicament

The present invention provides peptide sequences and compounds, capable i) stimulating neurite outgrowth; ii) modulating cell motility; ii) stimulating neural cell survival; iii) stimulating neural cell differentiation; iv) stimulating neural plasticity associated with memory and learning; v) modulating activity of a neurotrophin receptor of the Trk family receptors. Accordingly, the compounds may be useful for the stimulating neurite outgrowth, stimulating neural cell survival, stimulating neural plasticity, stimulating neural cell differentiation, modulating activity of a neurotrophin receptor of the Trk family receptors and modulating cell motility and for the manufacture of a medicament for the treatment of diseases and/or conditions, wherein said stimulating or said modulating is required. In some embodiments, the peptide sequences and compounds of the invention include isolated peptide fragments of neutrophins, such as NGF, NT-3 and NT-4/5, and/or NCAM.

Nerve growth factor (NGF) stimulates cholinergic function, improves memory and prevents cholinergic degeneration in animal models of injury, amyloid overexpression and aging, stimulates neurite outgrowth and neuronal survival (Tuszynski M H, et al. Nat. Med. 2005 June; 11 (6):551-5; Jakubowska-Dogru E, Gumusbas U Neurosci Lett. 2005 Jul. 1; 382(1-2):45-50; Walz R, et al Neurochem Res. 2005 February; 30(2):185-90; Hu Z et al. Neurobiol Dis. 2005 February; 18(1):184-92). Further, level of expression and activity of different Trk receptors has been correlated with cancer patient survival (Schramm A et al. Cancer Lett. 2005 May 24, PubMed Epub ahead of print) and invasive behaviour of cancer cells is dependent on both expression and activity of NCAM, Trk receptors and NGF (Chen-Tsai C P et al Dermatol Surg. 2004 July; 30(7):1009-16).

NCAM function has been shown to be important for a huge variety of normal and pathological conditions of different body systems and organs, in particular the nervous system (Sandi C. Nat Rev Neurosci. 2004 December; 5(12):917-30; Kiryushko D et al. Ann N Y Acad. Sci. 2004 April; 1014:140-54; Gniadecki R et al. Arch Dermatol. 2004 April; 140(4):427-36; Berezin V, Bock E. J Mol. Neurosci. 2004; 22(1-2):33-39).

Thus, a peptide sequence and/or compound of the invention may be used for prevention and/or treatment of a condition or disease, wherein the function of a neurotrophin or neurotrophin receptor, or function of NCAM plays an important role. Accordingly, the following non-limited examples of conditions and diseases may be contemplated wherein use of a peptide sequence and/or compound of the invention may have a beneficial effect in treatment or prevention thereof:
1) diseases and conditions of the central and peripheral nervous system such as or associated with postoperative nerve damage, traumatic nerve damage, impaired myelination of nerve fibers, postischaemic damage, e.g. resulting from a stroke, Parkinson's disease, Alzheimer's disease, Huntington's disease, dementias such as multiinfarct dementia, sclerosis, nerve degeneration associated with diabetes mellitus, disorders affecting the circadian clock or neuro-muscular transmission, and/or
2) mental diseases and disorders, such as a disorder of thought and/or mood, neuropsychiatric disorders including bipolar (BPD), genetically related unipolar affective disorders, delusional disorders, paraphrenia, paranoid psychosis, schizophrenia, schizotypal disorder, schizoaffective disorder, schizoaffective bipolar and genetically related unipolar affective disorders, psychogenic psychosis, catatonia, periodic bipolar and genetically related unipolar affective disorders, cycloid psychosis, schizoid personality disorder, paranoid personality disorder, bipolar and genetically related unipolar affective disorders related affective disorders and subtypes of unipolar affective disorder, and/or
3) diseases or conditions of the muscles including conditions with impaired function of neuro-muscular connections, such as after organ transplantation, or such as genetic or traumatic atrophic muscle disorders; and/or
4) diseases or conditions of various organs, such as degenerative conditions of the gonads, of the pancreas such as diabetes mellitus type I and II, of the kidney such as nephrosis and of the heart, liver and bowel, and/or
5) impaired ability to learn and/or impaired short-term and/or long-term memory.

In particular, the invention concerns treatment of normal, degenerated or damaged NCAM, Trk receptor and/or neurotrophin presenting cells.

The compounds of the invention are also capable of modulating cell motility, i.e. inhibiting cell motility. Thus, said compounds are also concerned by the invention for modulating cell motility, preferably for inhibiting of cell motility.

Thus, in this concern, a compound and medicament of the invention may be used for prevention and/or treatment of
1) cancer,
2) inflammatory disease,
3) allergic condition, and/or
4) neoangeogenesis.

The invention concerns cancer being any type of solid tumors requiring neoangiogenesis and any malignant cancer. In particular, the invention concerns cancer of the neural system.

A peptide sequence and/or compound of the invention may also be used for treating individuals having body damages due to alcohol consumption and for treating individuals suffering from prion diseases

Thus, it is an objective to use a peptide sequence, compound, and/or antibody for the manufacturing a medicament. A medicament of the invention may be used treatment any condition or disease wherein stimulating neurite outgrowth, stimulating neural cell survival, stimulating neural plasticity, stimulating neural cell differentiation, modulating activity of a neurotrophin receptor of the Trk family receptors and modulating cell motility is beneficial for the treatment. Non-limited examples of conditions and disease are described above.

The medicament of the invention may comprise an effective amount of one or more isolated peptide sequences, compounds or antibodies as described above, or it may be formulated as a pharmaceutical composition comprising an effective amount of one or more isolated peptide sequences, compounds or antibodies as described above and pharmaceutically acceptable additives. In some embodiments a medicament or pharmaceutical composition may comprise a combination of an effective amount of one or more isolated peptide sequences, compounds and/or antibodies as described above.

Thus, the invention in another aspect also concerns a pharmaceutical composition comprising at least one isolated peptide sequence, compound, and/or antibody of the invention.

A further aspect of the invention is a process of producing a pharmaceutical composition, comprising mixing an effective amount of one or more isolated peptide sequences, compounds or antibodies of the invention, or a pharmaceutical composition according to the invention with one or more pharmaceutically acceptable additives or carriers.

In one embodiment of the process as mentioned above, the compounds are used in combination with a prosthetic device, wherein the device is a prosthetic nerve guide. Thus, in a further aspect, the present invention relates to a prosthetic nerve guide, characterised in that it comprises one or more of the compounds or the pharmaceutical composition as defined above. Nerve guides are known in the art.

The invention also relates to the use a pharmaceutical composition comprising the compound of invention for the treatment or prophylaxis of any of the diseases and conditions mentioned in the application.

In some embodiments, the inventions relates to a pharmaceutical composition comprising an antibody capable of recognizing an epitope comprising the amino acid motif of the invention. Such pharmaceutical composition may also be useful in treatment of conditions and diseases described herein.

A medicament and/or pharmaceutical composition of the invention may suitably be formulated for oral, percutaneous, intramuscular, intravenous, intracranial, intrathecal, intracerebroventricular, intranasal or pulmonal administration.

Strategies in formulation development of medicaments and compositions based on the compounds of the present invention generally correspond to formulation strategies for any other protein-based drug product. Potential problems and the guidance required to overcome these problems are dealt with in several textbooks, e.g. “Therapeutic Peptides and Protein Formulation. Processing and Delivery Systems”, Ed. A. K. Banga, Technomic Publishing AG, Basel, 1995.

Injectables are usually prepared either as liquid solutions or suspensions, solid forms suitable for solution in, or suspension in, liquid prior to injection. The preparation may also be emulsified. The active ingredient is often mixed with excipients which are pharmaceutically acceptable and compatible with the active ingredient. Suitable excipients are, for example, water, saline, dextrose, glycerol, ethanol or the like, and combinations thereof. In addition, if desired, the preparation may contain minor amounts of auxiliary substances such as wetting or emulsifying agents, pH-buffering agents, or which enhance the effectiveness or transportation of the preparation.

Formulations of the compounds of the invention can be prepared by techniques known to the person skilled in the art. The formulations may contain pharmaceutically acceptable carriers and excipients including microspheres, liposomes, microcapsules, nanoparticles or the like.

The preparation may suitably be administered by injection, optionally at the site, where the active ingredient is to exert its effect. Additional formulations which are suitable for other modes of administration include suppositories, nasal, pulmonal and, in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

Other formulations are such suitable for nasal and pulmonal administration, e.g. inhalators and aerosols.

The active compound may be formulated as neutral or salt forms. Pharmaceutically acceptable salts include acid addition salts (formed with the free amino groups of the peptide compound) and which are formed with inorganic acids such as, for example, hydrochloric or phosphoric acids, or such organic acids as acetic acid, oxalic acid, tartaric acid, mandelic acid, and the like. Salts formed with the free carboxyl group may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred μg active ingredient per administration with a preferred range of from about 0.1 μg to 5000 μg per kilo body weight. Using monomeric forms of the compounds, the suitable dosages are often in the range of from 0.1 μg to 5000 μg per kilo body weight, such as in the range of from about 0.1 μg to 3000 μg per kilo body weight, and especially in the range of from about 0.1 μg to 1000 μg per kilo body weight. Using multimeric forms of the compounds, the suitable dosages are often in the range of from 0.1 μg to 1000 μg per kilo body weight, such as in the range of from about 0.1 μg to 750 μg per kilo body weight, and especially in the range of from about 0.1 μg to 500 μg per kilo body weight such as in the range of from about 0.1 μg to 250 μg per kilo body weight. In particular, when administering nasally smaller dosages are used than when administering by other routes. Administration may be performed once or may be followed by subsequent administrations. The dosage will also depend on the route of administration and will vary with the age and weight of the subject to be treated. A preferred dosage of multimeric forms would be in the interval 1 mg to 70 mg per 70 kg body weight.

For some indications a localised or substantially localised application is preferred.

For another application, intranasal application is preferred.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promote delivery of the active substance to its target.

In many instances, it will be necessary to administrate the formulation multiple times. Administration may be a continuous infusion, such as intraventricular infusion or administration in more doses such as more times a day, daily, more times a week, weekly, etc. It is preferred that administration of the medicament is initiated before or shortly after the individual has been subjected to the factor(s) that may lead to cell death. Preferably the medicament is administered within 8 hours from the factor onset, such as within 5 hours from the factor onset. Many of the compounds exhibit a long term effect whereby administration of the compounds may be conducted with long intervals, such as 1 week or 2 weeks.

In connection with the use in nerve guides, the administration may be continuous or in small portions based upon controlled release of the active compound(s). Furthermore, precursors may be used to control the rate of release and/or site of release. Other kinds of implants and well as oral administration may similarly be based upon controlled release and/or the use of precursors.

### 6. Treatment

Treatment by the use of the peptide sequences, compound(s) comprising thereof, antibodies, medicament(s) comprising thereof, and/or pharmaceutical composition(s) comprising thereof according to the invention is in one embodiment useful for inducing differentiation, modulating cell proliferation and motility, stimulating regeneration, survival and neuronal plasticity.

Thus, the treatment comprises treatment and/or prophylaxis of diseases or conditions of the central and peripheral nervous system, such as postoperative nerve damage, traumatic nerve damage, e.g. resulting from spinal cord injury, impaired myelination of nerve fibers, postischaemic damage, e.g. resulting from a stroke, multiinfarct dementia, multiple sclerosis, nerve degeneration associated with diabetes mellitus, neuro-muscular degeneration, schizophrenia, Alzheimer's disease, Parkinson's disease, or Huntington's disease.

Also, in relation to diseases or conditions of the muscles including conditions with impaired function of neuro-muscular connections, such as genetic or traumatic atrophic muscle disorders; or for the treatment of diseases or conditions of various or gans, such as degenerative conditions of the gonads, of the pancreas, such as diabetes mellitus type I and II, of the kidney, such as nephrosis the compounds according to the invention may be used for inducing differentiation, modulating proliferation, stimulate regeneration, neuronal plasticity.

The invention further concerns the use of the compound and/or pharmaceutical composition in the treatment of cancer. Regulation of motility of cancer cells is important for growth of tumors comprising cancer cells, invasion, angiogenesis and spreading thereof. Thus, the compound may advantageously be used as a medicament for the inhibiting the later processes in cancer prophylaxis and therapy

In yet a further embodiment the invention relates to use of the compound, and medicament comprising thereof and/or pharmaceutical composition comprising thereof for the stimulation of the short and/or long term memory and/or the ability to learn. Enhanced neurite outgrowth and establishment new neuronal connections are a part of the mechanism of memory consolidation. The peptide sequences and compounds comprising thereof may be useful for stimulation of both learn-term memory and short-term memory.

A compound, medicament and/or pharmaceutical composition of the invention may for example be used in the treatment of clinical conditions such as neoplasms such as malignant neoplasms, benign neoplasms, carcinoma in situ and neoplasms of uncertain behavior, diseases of endocrine glands, such as diabetes mellitus, psychoses, such as senile and presenile organic psychotic conditions, alcoholic psychoses, drug psychoses, transient organic psychotic conditions, Alzheimer's disease, cerebral lipidoses, epilepsy, general paresis (syphilis), hepatolenticular degeneration, Huntington's chorea, Jakob-Creutzfeldt disease, multiple sclerosis, Pick's disease of the brain, syphilis, Schizophrenic disorders, affective psychoses, neurotic disorders, personality disorders, including character neurosis, nonpsychotic personality disorder associated with organic brain syndromes, paranoid personality disorder, fanatic personality, paranoid personality (disorder), paranoid traits, sexual deviations and disorders, mental retardation, disease in the nervesystem and sense organs, cognitive anomalies, inflammatory disease of the central nervous system, such as meningitis, encephalitis, Cerebral degenerations such as Alzheimer's disease, Pick's disease, senile degeneration of brain, communicating hydrocephalus, obstructive hydrocephalus, Parkinson's disease including other extra pyramidal disease and abnormal movement disorders, spinocerebellar disease, cerebellar ataxia, Marie's, Sanger-Brown, Dyssynergia cerebellaris myocionica, primary cerebellar degeneration, such as spinal muscular atrophy, familial, juvenile, adult spinal muscular atrophy, motor neuron disease, amyotrophic lateral sclerosis, motor neuron disease, progressive bulbar palsy, pseudobulbar palsy, primary lateral sclerosis, other anterior horn cell diseases, anterior horn cell disease, unspecified, other diseases of spinal cord, syringomyelia and syringobulbia, vascular myelopathies, acute infarction of spinal cord (embolic) (nonembolic), arterial thrombosis of spinal cord, edema of spinal cord, subacute necrotic myelopathy, subacute combined degeneration of spinal cord in diseases classified elsewhere, myelopathy, drug-induced, radiation-induced myelitis, disorders of the autonomic nervous system, disorders of peripheral autonomic, sympathetic, parasympathetic, or vegetative system, familial dysautonomia [Riley-Day syndrome], idiopathic peripheral autonomic neuropathy, carotid sinus syncope or syndrome, cervical sympathetic dystrophy or paralysis. peripheral autonomic neuropathy in disorders classified elsewhere, amyloidosis, diseases of the peripheral nerve system, brachial plexus lesions, cervical rib syndrome, costoclavicular syndrome, scalenus anterior syndrome, thoracic outlet syndrome, brachial neuritis or radiculitis, including in newborn. Inflammatory and toxic neuropathy, including acute infective polyneuritis, Guillain-Barre syndrome, Postinfectious polyneuritis, polyneuropathy in collagen vascular disease, disorders affecting multiple structures of eye, purulent endophthalmitis, diseases of the ear and mastoid process, chronic rheumatic heart disease, ischaemic heart disease, arrhythmia, diseases in the pulmonary system, abnormality of organs and soft tissues in newborn, including in the nerve system, complications of the administration of anesthetic or other sedation in labor and delivery, diseases in the skin including infection, insufficient circulation problem, injuries, including after surgery, crushing injury, burns. Injuries to nerves and spinal cord, including division of nerve, lesion in continuity (with or without open wound), traumatic neuroma (with or without open wound), traumatic transient paralysis (with or without open wound), accidental puncture or laceration during medical procedure, injury to optic nerve and pathways, optic nerve injury, second cranial nerve, injury to optic chiasm, injury to optic pathways, injury to visual cortex, unspecified blindness, injury to other cranial nerve(s), injury to other and unspecified nerves. Poisoning by drugs, medicinal and biological substances, genetic or traumatic atrophic muscle disorders; or for the treatment of diseases or conditions of various organs, such as degenerative conditions of the gonads, of the pancreas, such as diabetes mellitus type I and II, of the kidney, such as nephrosis. Scrapie, Creutzfeldt-Jakob disease, Gerstmann-Straussler-Sheinker (GSS) disease.

According to invention a method of treatment and/or prevention of the above conditions and symptoms comprises a step of administering an effective amount of a peptide sequence and/or compound, and/or antibody and/or medicament, and/or pharmaceutical composition of the invention to an individual in need.

### 7. Examples

### Example 1

### Synthesis of Peptide Sequences

The peptides are synthesized on TentaGel resin with Rink amide linker (p-(R,S)-α-(1-(9H-fluoren-9-yl)-methoxyformamido)-2,4-dimethoxybenzyl)-phenoxyacetic acid (Novabiochem)) using Fmoc-protected amino acids (3 eq.). Coupling is performed for >60 min with TBTU (3 eq), HOBt (3 eq) and DIEA (4.5 eq) in a manual multicolumn apparatus. Fmoc is deprotected with 20% piperidine in DMF for 10 min. Synthesis of dendrimers of the peptides is accomplished by coupling Fmoc-Lys(Fmoc)-OH (Novabiochem) to the linker resin followed by Fmoc-deprotection of Fmoc group and further coupling of Fmoc-Lys(Fmoc)-OH is performed. After Fmoc-deprotection the synthesis of peptides is performed as above for monomeric peptides. Peptidyl resins are deprotected with 90% TFA, 5% H₂O, 3% EDT, 2% thioanisole, precipitated in dienyl ether, washed three times in diethyl ether, solubilised in 5% AcOH and lyophilized. Amino acid analysis is performed using Waters picotag and Waters 501 pump connected to WISP 712. Waters 600E equipped Waters 996 photodiode array detector is used for analytical and preparative HPLC on C18 columns (Delta-Pak 100 Å 15 um, Millipore). Maldi-MS may be done on a VG TOF Spec E, Fisions Instrument. The peptides are to be at least 95% pure as estimated by HPLC.

### Example 2

### Stimulation of Neurite Outgrowth by NCAM HBP

Co-Culture Fibroblasts with or without Expression of NCAM and Primary CGNs

Genetically modified fibroblasts (mouse fibroblastoid L-cells) with or without NCAM expression are seeded in eight-well LabTek tissue culture chamber slides (Nunc, Roskilde, Denmark) at a density 7×10⁴ of cells/well and maintained at 37° C. and 5% CO₂ in DMEM with 10% HS, 100 U/ml penicillin and 100 μg/ml streptomycin. 24 h after plating the medium is removed and CGNs (10⁴ cells/well) are seeded on monolayers of fibroblasts with or without NCAM expression in Neurobasal-A medium containing 100 U/ml penicillin, 100 μg/ml streptomycin, 1% glutamax and supplemented with B27, and grown in co-culture for 24 hours, afterwards neurite outgrowth is measured. CGNs are prepared from postnatal day 3-4 rat pups according to Rønn, L. C., Doherty, P., Holm, A., Berezin, V., Bock, E. (2000). J. Neurochem. 75(2):665-71.

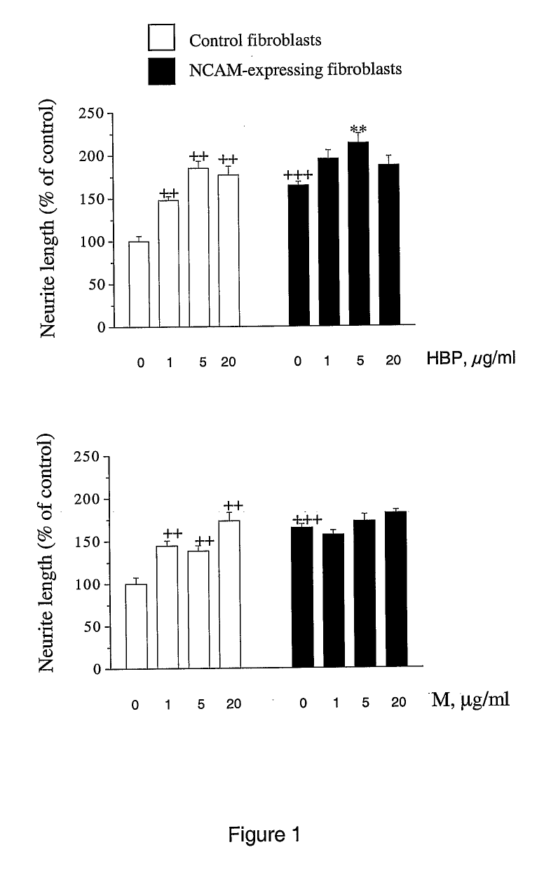

To evaluate the effect of the heparin binding peptide (HBP) of NCAM (KGRDVILKKDVRFI) (SEQ ID NO: 1) (amino acids 154-167 of IglI NCAM (SwissProt P13596). CGNs are seeded on fibroblasts with or without NCAM expression in the presence of different concentrations of HBP or the peptide M (KGRDVILAKDVRVI) (SEQ ID NO: 2), in which Lys-161 and Phe-166 (corresponding to the residues of the HBP sequence) are substituted for Ala and Val, respectively. From FIG. 1 it appears that NCAM expressing fibroblasts significantly promote neurite outgrowth CGNs as compared to control fibroblasts without NCAM expression, and HBP in a dose-dependent manner increases neurite outgrowth of CGNs grown on fibroblasts expressing NCAM as well as on fibroblasts without NCAM expression. In contrast, the peptide M does not affect the neurite outgrowth response from CGNs grown on NCAM expressing fibroblasts, but it still stimulates neurite outgrowth of CGNs grown on fibroblasts without NCAM expression.

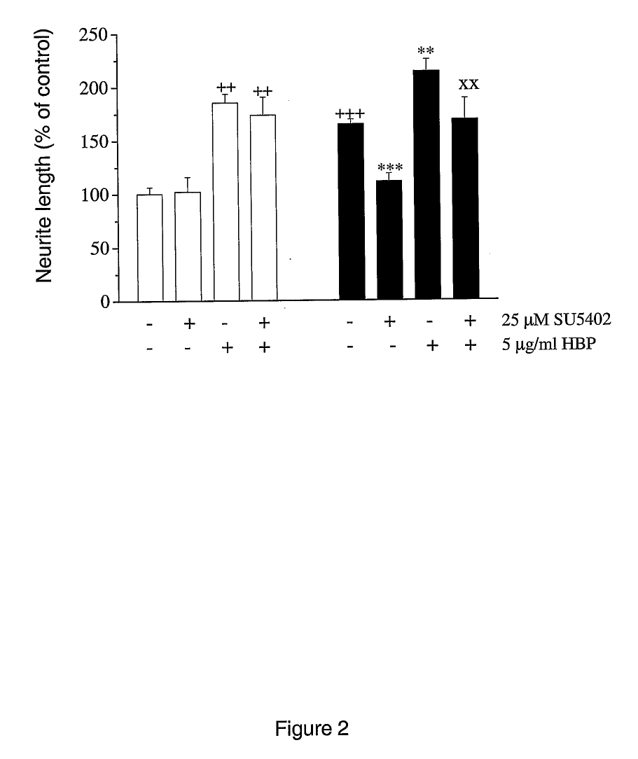

From FIG. 2 it appears that the neuritogenic effect of HBP on CGNs grown on control fibroblasts is not blocked by treatment with SU5402, an inhibitor of the fibroblast growth factor receptor (FGFR), and NCAM mediated neurite outgrowth in co-cultures of CGNs with NCAM expressing fibroblasts is only partially inhibited, however SU5402 blocks NCAM mediated neurite outgrowth in the absence of HBP. This indicates that HBP induced neurite outgrowth is dependent on at least two mechanisms: 1. dependent on activation of NCAM-FGFR signaling, when trans NCAM homophilic interaction is involved, and 2. independent on FGFR signaling, when there is no trans NCAM homophilic interaction.

### Primary Culture of Single CGNs Neurons

Primary cerebellar granule neurons (CGNs) are prepared from postnatal day 3-4 rat pups according to Rønn, L. C., Doherty, P., Holm, A., Berezin, V., Bock, E. (2000). J. Neurochem. 75(2):665-71. Dissociated cerebella cells are grown at 37° C. and 5% CO₂ in Neurobasal-A medium containing 20 mM Hepes, 100 U/ml penicillin, 100 μg/ml streptomycin, 1% glutamax and supplemented with B27 (all from Gibco BRL). For testing the effect of the peptides, CGNs are seeded in eight-well LabTech tissue chamber slides with a growth surface of Permanox plastic (Nunc) at a density of 10⁴ cells/well in Neurobasal-A medium containing 100 U/ml penicillin, 100 μg/ml streptomycin, 1% glutamax and supplemented with B27. When treated with Heparinase III (1 U/ml), the enzyme is added to the culture medium immediately after plating. CGNs are treated with the enzyme for 1 h before addition of different concentrations (1 μg/ml, 5 μg/ml, and 20 μg/ml) of the peptides. After 24 h of culture, cells are fixed with paraformaldehyde, immunostained with primary mouse antibodies against CD90 (Thy-1) (1:200) (Caltag Laboratories, USA) (PC12-E2 cells) or GAP-43 (1:1000) (Chemicon, USA) (CGNs) and secondary Alexa Fluor®488 goat antibody against mouse IgG (1:1000) (Molecular Probes, Netherlands), and the length of neurites is measured by a stereological method as previously described (Ronn, L. C., Ralets, I., Hartz, B. P., Bech, M., Berezin, A., Berezin, V., Møller, A., Bock, E (2000). J Neurosci Methods. 100(1-2), 25-32).

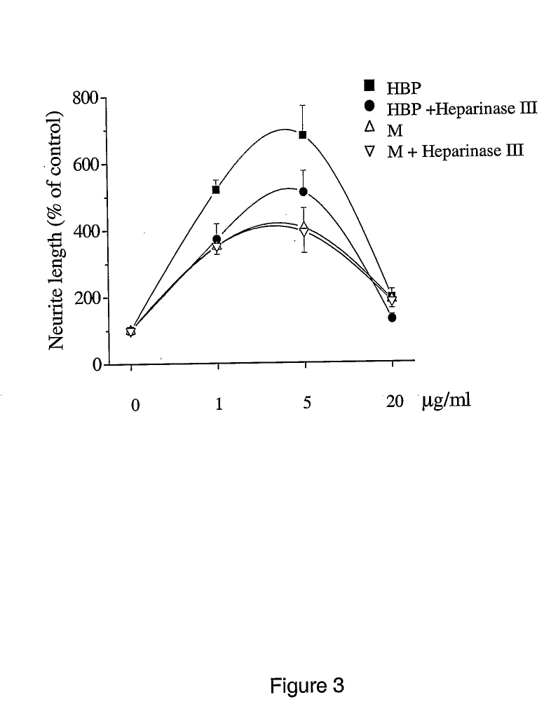

FIG. 3 demonstrates the effect of NCAM HBP (SEQ ID NO:1) on single primary CGNs, grown in culture in the presence or absence of heparinase III, the enzyme, which is known to specifically cleave heparan sulfate mainly into disaccharides. HBP immobilized on tissue culture plastic is capable induce neurite outgrowth in CGNs in a dose-dependent manner with a maximal effect of 680% at a coating concentration of 5 μg/ml. The peptide M had maximal effect of 400% at a coating concentration of 5 μg/ml. Treatment with heparinase III reduced the effect of HBP, but not the M peptide. These results suggest that the neurite outgrowth stimulating activity of NCAM HBP is dependent on two targets acting in concert, a heparan sulfate sensitive and a heparan sulfate insensitive target.

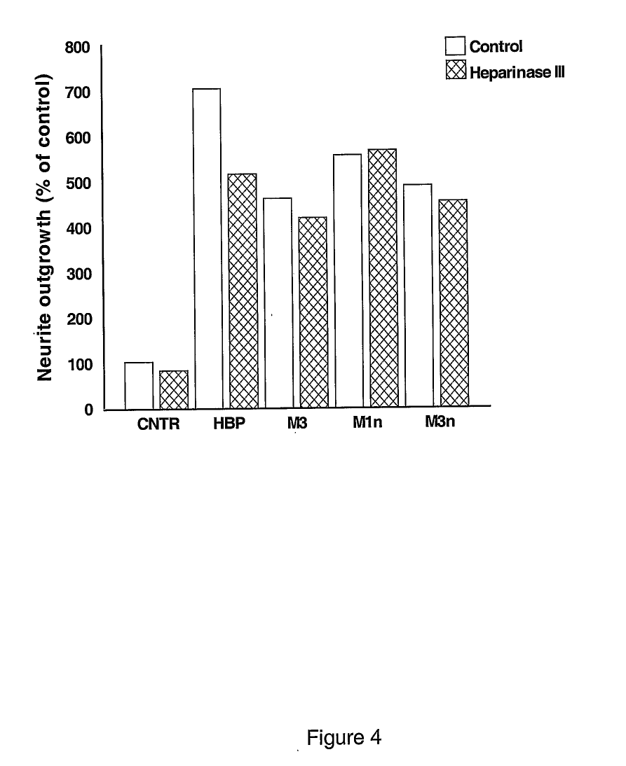

FIG. 4 demonstrates the neuritogenic effect of peptides: HBP (KGRDVILKKDVRFI) (SEQ ID NO: 1), peptide M (KGRDVILAKDVRVI) (SEQ ID NO: 2), peptide M1n (KGRDVILNNDVRFI) (SEQ ID NO: 3), and peptide M3n (KGRDVILNNQVRFI) (SEQ ID NO: 4) in the presence and absence of heparinase III. All peptides have a very strong neuritogenic activity reflected by a 4-7 fold increased neurite length of treated neurons. Heparin binding activity of NCAM HBP (SEQ ID NO: 1) is not a prerequisite for stimulation of neurite outgrowth by the peptide, however a neuritogenic potential of the sequence is stronger in the presence of heparan sulphate.

### Example 3

### Modulating of Motility of Fibroblasts by NCAM HBP

For evaluation the effect of the peptides on cell motility, confluent cultures of fibroblasts with or without NCAM expression are dislodged with 0.5 mg/ml of trypsin and 0.75 mM EDTA in a modified Puck's saline (Gibco BRL), seeded in six-well tissue culture plates (Nunc) at a density of 4×10³ cells/cm² and grown for 24 h at 37° C. and at 5% CO₂.

Random cell motility was evaluated by use of time-lapse video-recording and image analysis as described previously (Walmod, et al. (1998). Cell Motil Cytoskeleton. 40(3), 220-37). Briefly, tissue culture dishes tightly sealed with adhesive tape are placed on a thermostatically controlled stage (Lincam Scientific Instruments Ltd., UK) mounted on a Nikon Diaphot 300 inverted microscope (Nikon, Denmark). The temperature inside the plexiglas incubator mounted around the microscopic stage is maintained at 37° C. using a thermostatically controlled heating fan (DFA, Denmark). A motorized stage mounted on the microscope, allows simultaneous recordings from many (50-100) different microscopic fields. Video-recordings of live cells is made using a black and white CCD video camera (Burle, Lancaster, Pa.) attached to the microscope. Automated 768×576-pixel image acquisition and storage are performed with phase contrast optics using a 10× objective at 15 min intervals during 4 h using the software PRIMA (Protein Laboratory, Copenhagen, Denmark).

Evaluation of cell motility and morphology is performed using the image processing software PRIMA. The positions of individual cells are determined by manual marking of the centers of the nuclei. The track of a single cell is defined as a sequence of coordinates of nuclear centers at different time points. The parameters of mean squared cell speed (S_{τ}), rate of diffusion (R), and locomotive index (LI) are determined according to Walmod, et al. (2000) (Methods Mol. Biology. 161, 59-83) as described below.

### Analysis of Cell Motility

The displacement of a cell is the Euclidean distance between two points corresponding to the initial and final positions of the cell. Determinations of cell positions were performed with a constant time period, between successive observations. The mean squared cell displacement of a cellular population after a given time (t₁) of observation was calculated as

$$〈 {d}^{2} ( {t}_{i} ) 〉 = \frac{1}{N ( k - i + 1 )} {\sum }_{m = 1}^{N} {\sum }_{s = 1}^{k} \sqrt{{( x ( {t}_{s} ) - x ( {t}_{s - 1} ) )}^{2} + {( y ( {t}_{s} ) - y ( {t}_{s - 1} ) )}^{2}}$$

where i is the observation number (i=0, 1, 2, . . . k), k is the total number of observations minus one, tᵢ is the time interval between the initial observation (t₀) and the observation number i (tᵢ=i×τ), xₘ(tᵢ) and yₘ(tᵢ) are the coordinates of cell number m at the time point tᵢ, and N is the total number of cells. The rate of diffusion, R, was estimated by plotting the mean squared displacement, <d²>, against time with subsequent curve fitting to the equation:

[symbol]

d²(t₁) [symbol] =R(τ−P(1−e^{-τ/P}))

where P is the persistence time in direction (23). The mean cell speed, Sτ, was calculated as the ratio of the mean displacement of cells (<dτ>) to the time interval τ. This was done according to the equation:

$$\begin{matrix}〈 S τ 〉 = \frac{〈 {d}_{τ} 〉}{τ} \\ = \frac{\frac{1}{N \cdot k} {\sum }_{m = 1}^{N} {\sum }_{s = 1}^{k} \sqrt{{( {x}_{m} ( {t}_{s} ) - {x}_{m} ( {t}_{s - 1} ) )}^{2} + {( {y}_{m} ( {t}_{s} ) - {y}_{m} ( {t}_{s - 1} ) )}^{2}}}{τ}\end{matrix}$$

The mean cell-path-length, [symbol] L [symbol] , for a sample of a population of cells at a given time of observation, was calculated as:

$$〈 L 〉 = \frac{1}{N} {\sum }_{m = 1}^{N} {\sum }_{s = 1}^{k} \sqrt{{( {x}_{m} ( {t}_{s} ) - {x}_{m} ( {t}_{s - 1} ) )}^{2} + {( {y}_{m} ( {t}_{s} ) - {y}_{m} ( {t}_{s - 1} ) )}^{2}}$$

The locomotive index, LI, was calculated as the ratio of the mean cell displacement (<d>) and the mean cell-path-length:

$$LI = \frac{< d >}{< L >}$$

LI was used as a measure of directional persistence of cells. Cells moving in perfectly straight lines will have an LI of one, whereas a lower directional persistence will result in lower LI-values. For randomly moving cells LI has been demonstrated to exhibit a statistically significant correlation to the persistence time of direction (P), a parameter, reflecting the average time between significant changes in the directional movement of a single cell

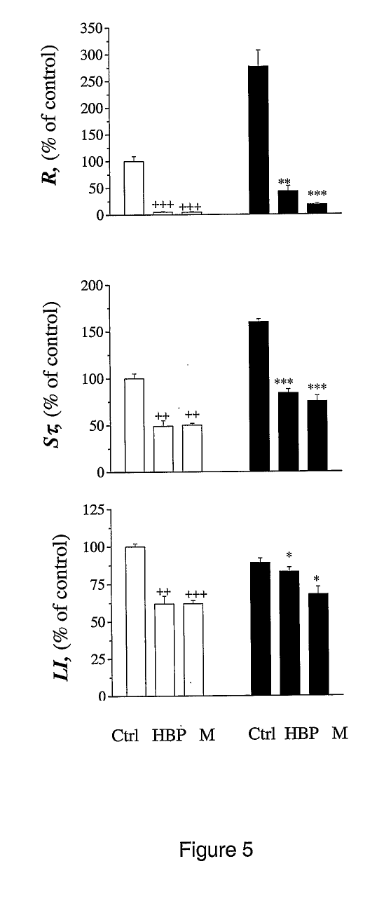

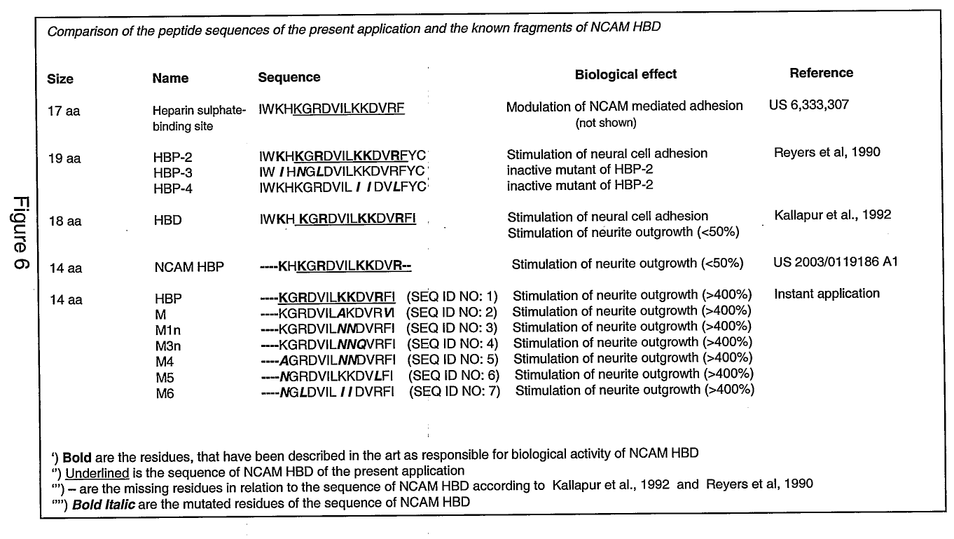

From FIG. 5 it appears, that the NCAM expressing fibroblasts moves much faster than the control fibroblasts (275%) as reflected by a higher rate of diffusion (R) of NCAM positive fibroblasts as compared to fibroblasts without NCAM expression. Treatment of both NCAM expressing fibroblast and control fibroblasts with HBP results in a clear inhibition of cell motility as reflected by a pronounced decrease of R, Sτ, and LI. The peptide M (SEQ ID NO: 2) has an inhibitory effect on the motile behaviour of fibroblasts similar to the effect of HBP. These results indicate that HBP inhibits fibroblast motility independent of NCAM expression and possibly independent of heparin binding. The latter supports the suggestion that the neurite outgrowth stimulating activity of NCAM HBP contains a heparan sulfate insensitive component.

### Example 4

### Analysis of Peptide Sequences of the Invention

FIG. 6 presents the results of comparative analysis of the peptide sequences of the invention (identified in SEQ ID NO: 1-7) and fragments of NCAM HBD which have been described in the art. From the figure it appears that 1) peptide fragments of the present application, namely HBP, M, M1n, M3n, M4, M5 and M6, are very potent stimulators of neurite outgrowth (treatment of neurons with the peptides in vitro lead to more then 400% stimulation of neurite outgrowth comparing to control (untreated) neurons), whereas the known in the art fragments of NCAM HBD are much less potent (the reported stimulation is less then 50%). Interestingly, mutation of the amino acid residues reported to be important for the biological activity of NCAM HBP does not influence the activity of the mutant peptides pointing thus to the presence another structure in the peptide sequences which is responsible for their biological activity such as stimulating neurite outgrowth and cell motility. Comparison of the amino sequences of 14 amino acid peptide fragments of NCAM HBD of the present invention and a 14 amino acid long peptide of US 2003/0119186 reveals a substantial difference between the sequences of the fragments and the sequence of the peptide. The peptide has two additional negatively charged amino acid residue on the N-terminus and lacks two hydrophobic residues on the Cterminus. Another sequence reported to have a stimulatory effect on neurite outgrowth (Kallapur et al., 1992) (see FIG. 6) is 18 amino acids long and comprises both the C-terminal hydrophobic residues and the additional basic residues. The sequence is much less potent as a stimulant then the sequences of the application.

Taken in account the above analysis and the threshold of stimulatory effect of the peptide sequences on neurite outgrowth which is comparable with the effect of neurotrophins that are well-known known major stimulators of neural cell differentiation and neurite outgrowth, the authors of the present invention did compare the sequences of the peptides of the invention to the sequences of neurotrophins, in particular to the sequences of NGF, NT-3, NT-4/5 and BDNF, and thus identified a novel neurotrophin receptor Trk binding motif.

Neurotrophins bind to and activate two types of receptors: p75^{NTR} receptor, activation of which is associated with induction of neuronal cell apoptosis, and receptors of the Trk family, activation of which is associated with stimulation of neurite outgrowth. Surprisingly the peptides sequences of the invention appeared to have a very high homology a fragment of the NGF sequence which is located in beta-hairpin loop 1 of the protein which comprises the amino acid motif TDIKGKE, which is reported to be crucial for binding NGF to receptor p75^{NTR} (Williams et al, J Biol Chem 280:5862-69, 2005; Ibanez et al., Cell 69:329-341, 1992; He and Garcia, Science 304:870-875, 2004), but the peptide sequences of the invention do not comprise the amino acid motif RGE reported to be important for Trk receptor binding and activating (Williams et al, J Biol Chem 280:5862-69, 2005; WO2005025514).

However, the authors of the present invention show herein a direct binding of HBP peptide to Trk B receptor and claim a novel Trk receptor binding amino acid motif which is described in detail above. The amino acid motif disclosed in the application points to the amino acid residues of the peptide sequences of the invention which are crucial for their neuritogenic activity. Accordingly, the sequence of NCAM HBD peptide of US 2003/0119186, which lacks an important the C-terminal hydrophobic residue, is not capable to stimulate neurite outgrowth to the same extend as the peptide sequences of the invention. From the other hand, the negatively charged residues on the N-terminus seem not to be essential for binding to Trk receptor, as the corresponding sequence form BDNF (SEQ ID NO: 11 of the present application), which is a natural ligand of Trk B receptor, does not have these residues. The latter may explain a decreased effect of sequence of NCAM HBP comprising these residues (Kallapur et al., 1992) on neurite outgrowth.

Thus, the authors of the present invention identify and claim herein the formula of a peptide sequence which is most effective for binding Trk receptor resulting in activation of the receptor.

### Example 5

### Binding NCAM HBP to Trk B Receptor

Recombinant the Ig2 module of NCAM was prepared using in the yeast P. pastoris expression system (Invitrogen, USA) as previously described (Kiselyov et al., Biol. Chem. 272, 10125-10134, 1997). Recombinant Trk B receptor was purchased from RD Systems (USA).

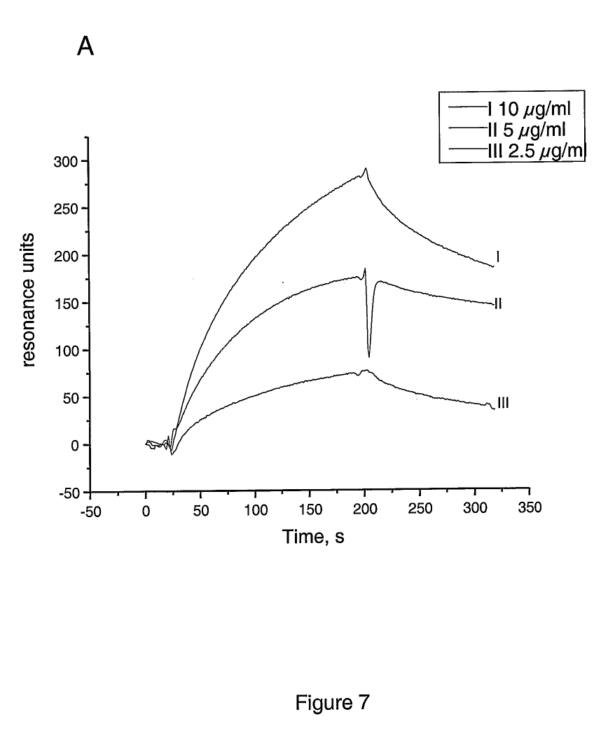

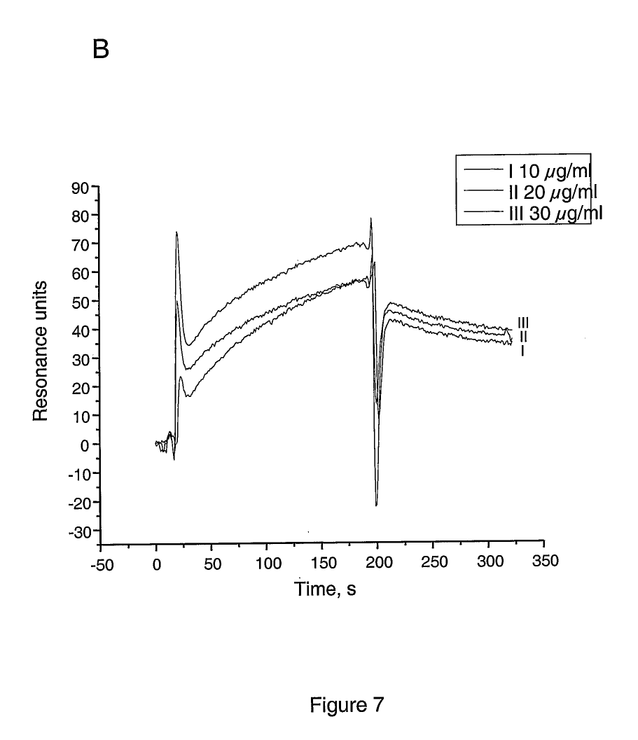

Binding analysis was performed using a BIAcoreX instrument (Biosensor AB) at 25° C. using 10 mM sodium phosphate pH 7.4, 150 mM NaCl as running buffer. The flow-rate was 5 μl/min. Data were analysed by non-linear curve-fitting using the manufacture's software. The HBP or recombinant Ig 2 module of NCAM were immobilized on a sensor chip CM5 using amine coupling kit (Biosensor AB) as follows: 1) the two halves of the chip (designated Fc1 and Fc2) were activated by 20 μl activation solution; 2) the protein was immobilized on Fc1 using 12 μl 20 μg/ml protein in 10 mM sodium phosphate buffer pH 6.0; 3) Fc1 and Fc2 were blocked by 35 μl blocking solution. Binding of Trk B to the immobilized HBP or the Ig module II of NCAM was studied as follows: Trk B was injected simultaneously into Fc1 (with the immobilized HBP or Ig module II of NCAM) and Fc2 (with nothing immobilized). The curve representing unspecific binding of the compound to the surface of Fc2 was subtracted from the curve representing binding of Trk B to the immobilized protein (HBP or the Ig 2 module of NCAM) and the surface of Fc1. The resulting curve demonstrating the recorded binding is demonstrated in FIG. 7. HBP NCAM binds to Trk B receptor both as the isolated peptide sequence and integrated sequence of the Ig 2 module of NCAM with K_{D}˜10⁻⁸-10⁻⁷ M and K_{D}˜10⁻⁹-10⁻⁸ M correspondingly.

1. An isolated contiguous peptide sequence consisting of 6 to 13 amino acid residues, said peptide sequence comprising the amino acid motif
G-x^{a}-D/E/Q/T-V-x^{b}-V/L
Wherein
x^{a} is any amino acid residue,
x^{b} is I, T, M or E.

2. The isolated contiguous peptide sequence according to claim 1, wherein x^{a} is a basic amino acid residue.

3. The isolated contiguous peptide sequence according to claim 1, wherein x^{a} is L.

4. The isolated contiguous peptide sequence according to claim 1, wherein x^{a} is G.

5. The isolated contiguous peptide sequence according to claim 1, said peptide sequence is defined by the formula (I)
x₀-x₁-x₂-x₃-x₄-x₅-x₆-x₇-x₈-x₉-x₁₀-x₉-x₉-x₁₀-x₁₁-x₁₂-x₁₃
Wherein
x₀ is K, R, N, A or a bond x₁ is G,
x₂ is a basic or hydrophobic amino acid residue,
x₃ is E or D,
x₄ is V,
x₅ is a hydrophobic amino acid residue,
x₆ is V or L,
x₇ is any amino acid residue,
x₈ is any amino acid residue,
x₉ is E, D, K or Q,
x₁₀ is V or L,
x₁₁ is any amino acid residue,
x₁₂ is a hydrophobic amino acid residue or T,
x₁₃ is I, N, S, G, A or a bond.

6. (canceled)

7. (canceled)

8. The isolated peptide sequence according to claim 5, wherein x₇ and x₈ are independently selected from K, R, N, I, L, G, E or A.

9. The isolated peptide sequence according to claim 8, wherein x₇ is L.

10. The isolated peptide sequence according to claim 5, wherein x₈ is G.

11. The isolated peptide sequence according to claim 5, wherein x₈ is E.

12. The isolated peptide sequence according to claim 5, wherein x₁₁ is R, K, L, N or P.

13. The isolated peptide sequence according to claim 5, wherein x₁₂ is a hydrophobic amino acid residue and selected from F, V, I, T or A.

14. The isolated contiguous peptide sequence according to claim 1, wherein said peptide sequence is defined by the formula (II) x₀-G-x₂-D/E-V-I-L-x₇-x₂-x₉-V-x₁₁-x₁₂-x₁₃ wherein x₀ is an amino acid residue selected from K, R, A or N, x₂ is an amino acid residue selected from R or L, x₇ is an amino acid residue selected from K, A, N or L, x₈ is an amino acid residue selected from K, N or L, x₉ is an amino acid residue selected from D or Q, x₁₁ is an amino acid residue selected from R or L, x₁₂ is an amino acid residue selected from V or F, x₁₃ is an amino acid residue selected from I, L or V.

15. The isolated contiguous peptide sequence according to claim 5, said peptide sequence is defined by the formula (III) x₀-G-x₂-x₃-V-x₅-V-L-G/E-x₉-V/L-x₁₀-x₁₁-x₁₂-x₁₃ wherein x₀, x₂, x₃, x₅, x₉, x₁₀, as defined in claim 5, x₁₁ is N, K or P, x₁₂ is I, T, A or V and x₁₃ is S, A, N or G.

16. (canceled)

17. The isolated peptide sequence according to claim 5, wherein x₁₁ is R, N, P or L.

18. The isolated peptide sequence according to claim 5, wherein the amino acid sequence comprises the amino acid sequence motif as defined in claim 1.

19. The isolated peptide sequence according to claim 1, wherein the amino acid sequence is KGRDVILKKDVRFI (SEQ ID NO: 1).

20. The isolated peptide sequence compound according to claim 1, wherein the amino acid sequence is KGRDVILAKDVRVI (SEQ ID NO: 2).

21. The isolated peptide sequence according to claim 1 wherein the amino acid sequence is KGRDVILNNDVRFI (SEQ ID NO: 3).

22. The isolated peptide sequence according to claim 1, wherein the amino acid sequence is KGRDVILNNQVRFI (SEQ ID NO: 4).

23. The isolated peptide sequence according to claim 1, wherein the amino acid sequence is AGRDVILNNDVRFI (SEQ ID NO: 5).

24. The isolated peptide sequence according to claim 1, wherein the amino acid sequence is NGRDVILKKDVLFI (SEQ ID NO: 6).

25. The isolated peptide sequence according to claim 1, wherein the amino acid sequence is NGLDVILIIDVRFI (SEQ ID NO: 7).

26. The isolated peptide sequence according to claim 1, wherein the amino acid sequence is KGKEVMVLGEVNIN (SEQ ID NO: 8).

27. The isolated peptide sequence according to claim 1, wherein the amino acid sequence is RGHQVTVLGEIKTG (SEQ ID NO: 9).

28. The peptide according to the preceding claim 1, wherein the amino acid sequence is RGREVEVLGEVPAA (SEQ ID NO: 10).

29. The peptide according to the preceding claim 1, wherein the amino acid sequence is SGGTVTVLEKVPVS (SEQ ID NO: 11).

30. The isolated peptide sequence according to claim 1, wherein the peptide sequence is a fragment, variant or homologue of any of the sequences selected from SEQ ID NOS: 1-10, said fragment, variant or homologue comprising the motif as defined in claim 1.

31. The isolated peptide sequence according to claim 1, wherein said peptide sequence is capable of binding to a neurotrophin receptor of the Trk family receptors comprising Trk A, Trk B and Trk C.

32. The isolated peptide sequence according to claim 31, wherein the receptor is Trk A or Trk B.

33. The isolated peptide sequence according to claim 1, wherein said peptide sequence is capable of stimulating neuronal cell differentiation.

34. The isolated peptide sequence according to claim 1, wherein the peptide sequence is capable of stimulating neurite outgrowth.

35. The isolated peptide sequence according to claim 1, wherein said peptide sequence is capable of stimulating neuronal cell survival.

36. The isolated peptide sequence according to claim 1, wherein said peptide sequence is capable of modulating cell motility.

37. The isolated peptide sequence according to claim 1, wherein the peptide sequence is capable of inhibiting cell motility.

38. The isolated peptide sequence according to claim 1, wherein said peptide sequence is capable of stimulating neural plasticity associated with learning and/or memory.

39. The isolated peptide sequence according to claim 19, wherein the peptide sequence is a peptide fragment of the neural cell adhesion molecule (NCAM).

40. The isolated peptide sequence according to claim 26, wherein the peptide sequence is a peptide fragment of nerve growth factor (NGF).

41. The isolated peptide sequence according to claim 27, wherein the peptide sequence is a peptide fragment of neurotrophin-3 (NT-3).

42. The isolated peptide sequence according to claim 28, wherein the peptide is a peptide fragment of neurotrophin-4/5 (NT-4/5).

43. The isolated peptide sequence according to claim 29, wherein the peptide is a peptide fragment of brain-derived neurotrophic factor (BDNF).

44. A compound comprising an isolated peptide sequence according to claim 1.

45. The compound according to claim 44, wherein the peptide sequence is formulated as monomer consisting of the single copy of an individual peptide sequence.

46. The compound according to claim 44, wherein the peptide sequence is formulated as a multimer consisting of two or more copies of an individual peptide sequence, such as a dimer or tetramer of an individual peptide sequence.

47. The compound of claim 46, wherein the multimer is a dendrimer of two or more individual peptide sequences.

48. The compound of claim 46, wherein the multimer is a dimer comprising two identical peptide sequences.

49. The compound of claim 46, wherein the multimer is a dimer comprising two different peptide sequences.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. An antibody capable of recognizing and binding to an epitope comprising the motif according to claim 1.

70. A pharmaceutical composition comprising an antibody according to claim 69.

71. A Method of treatment, wherein the treatment is of a condition or disease wherein stimulating neural cell differentiation, neural cell survival, neural cell plasticity, stimulating learning and/or memory, modulating cell motility or modulating the activity of a neurotrophin receptor of the Trk family receptors is a part of said treatment comprising administering an effective amount of a peptide according to claim 1.

72. Method of treatment, wherein the treatment is of a condition or disease of the central and peripheral nervous system, comprising administering an effective amount of a peptide according to claim 1.

73. Method of treatment, wherein the treatment is of a condition or disease selected from postoperative nerve damage, traumatic nerve damage, impaired myelination of nerve fibers, postischaemic damage, nerve degeneration associated with diabetes mellitus, disorders affecting the circadian clock or neuro-muscular transmission, comprising administering an effective amount of a peptide according to claim 1.

74. Method of treatment, wherein the treatment is of a condition or disease selected from conditions or diseases of the muscles including conditions with impaired function of neuro-muscular connections, or genetic or traumatic atrophic muscle disorders, comprising administering an effective amount of a peptide according to claim 1.

75. Method of treatment, wherein the treatment is of a condition or disease associated with neoangiogenesis, tissue remodelling and/or increased motility of the cells, comprising administering an effective amount of a peptide according to claim 1.

76. The method according to claim 75, wherein the disease is cancer.

77. The method according to claim 76, wherein the cancer is any cancer involving neoangiogenesis.

78. The method according to claim 76, wherein the cancer is a cancer of neural system.

79. The method according to claim 71, wherein the condition or disease is an impaired ability to learn and/or impaired memory.

80. The method according to claim 71, wherein the condition or disease is Parkinson's disease, Alzheimer's disease, Huntington's disease or dementia such as multiinfarct dementia.

81. The method according to claim 71, wherein the condition or disease is a mental disease, neuropsychiatric disorders including bipolar (BPD), genetically related unipolar affective disorders, delusional disorders, paraphrenia, paranoid psychosis, schizophrenia, schizotypal disorder, schizoaffective disorder, schizoaffective bipolar and genetically related unipolar affective disorders, psychogenic psychosis, catatonia, periodic bipolar and genetically related unipolar affective disorders, cycloid psychosis, schizoid personality disorder, paranoid personality disorder, bipolar and genetically related unipolar affective disorders related affective disorders and subtypes of unipolar affective disorder.

82. Method of treatment, wherein the treatment is of a condition or disease associated with body damages due to alcohol consumption, comprising administering an effective amount of a peptide according to claim 1.

83. Method of treatment, wherein the treatment is of prion diseases, comprising administering an effective amount of a peptide according to claim 1.

84. A medicament comprising an individual peptide sequence according to claim 1 or a compound according to claim 44.

85. A pharmaceutical composition comprising an effective amount of a medicament according to claim 84.

86. Method of treatment comprising administering to an individual in need thereof an effective amount of a peptide sequence according to claim 1.

87. Method of stimulating neural cell differentiation, neural cell survival, neural cell plasticity and/or modulating cell motility, said method comprising administering an individual peptide sequence according to claim 1.
